(19) United States
(12) Patent Application Publication
(10) Pub. No.: US 2026/0315629 A1
(43) Pub. Date: Oct. 15, 2026

(54) MOLECULAR-MRI USING CCR2-TARGETED MICELLES FOR DETECTION OF LYMPH NODE METASTASES

(71) Applicant: UNIVERSITY OF SOUTHERN CALIFORNIA, Los Angeles, CA (US)

(72) Inventors: Eun Ji Paige CHUNG YOO, Los Angeles, CA (US); Noah Tom TRAC, Los Angeles, CA (US)

(73) Assignee: UNIVERSITY OF SOUTHERN CALIFORNIA, Los Angeles, CA (US)

(21) Appl. No.: 19/476,459

(22) PCT Filed: Apr. 17, 2024
(86) PCT No.: PCT/US2024/024999

§ 371 Date: Oct. 17, 2025

Related U.S. Application Data
(60) Provisional application No. 63/459,807, filed on Apr. 17, 2023.

(51) Int. Cl. A61K 49/18; A61K 49/08; B82Y 5/00; B82Y 15/00

(52) U.S. Cl. CPC A61K 49/1866; A61K 49/085; B82Y 5/00; B82Y 15/00

(57) ABSTRACT

An MRI contrast delivery system includes nanoparticles that are each equipped with targeting peptides for CCR2 receptors and an MRI contrast moiety. These components are conjugated to the nanoparticles, which are made from hydrophobic lipids. This design enables the nanoparticles to “hitchhike” on monocytes, leveraging their natural migration pathways to efficiently target metastatic lymph nodes (LNs). This targeted delivery approach significantly enhances the specificity and sensitivity of the MRI signals, improving the imaging of metastatic sites and potentially aiding in more accurate diagnosis and assessment of cancer progression.

### CROSS-REFERENCE TO RELATED APPLICATIONS

This application claims the benefit of U.S. provisional application Ser. No. 63/459,807 filed Apr. 17, 2023, the disclosure of which is hereby incorporated in their entirety by reference herein.

### TECHNICAL FIELD

In at least one aspect, the present invention is related to targeted micelles for the detection of lymph node metastases.

### BACKGROUND

Nearly 20 million patients are diagnosed with cancer each year globally. (1) Given that an estimated 90% of solid cancers metastasize through the lymph nodes (LNs), the accurate detection of LN metastasis is critical in cancer staging, treatment, and patient outcome. (2) For example, melanoma progression from regional LN to distant metastasis is associated with a 55% decrease in patient 5-year survival, demonstrating the importance of the detection and treatment of LN metastasis. (3) In addition, early detection of metastatic LN recurrence has been reported to increase patient survival from 14% to 36% by identifying patients that would benefit from adjuvant chemo- and immunotherapies. (4)

Despite the clear significance of metastatic LN detection in patient outcomes, LN biopsies remain the clinical standard for detecting LN metastasis during initial cancer screening. However, because biopsies only evaluate regional LNs near the primary tumor, they are subject to false-negative rates as high as 20%. (5) Moreover, LN biopsies are invasive and can lead to patient morbidities including infection, lymphedema, and thrombosis. (6,7) In response to these shortcomings, noninvasive imaging tools have been explored for detecting LN metastasis but have yet to gain clinical adoption. Positron emission tomography/computed tomography (PET/CT) can combine anatomical and functional imaging but has low sensitivity for detecting LN metastasis (~11% of metastases detected) and requires the use of radioactive tracers and ionizing radiation. (8) Near-infrared fluorescence (NIRF) imaging does not require radiation but is associated with poor tissue penetration (<2 cm), limiting its application to superficial imaging. (9) Ultrasonography (US) is a low-cost imaging modality that can detect larger LN metastases but is relatively insensitive for detecting micrometastases smaller than 1 cm (~7% of micrometastases detected). (10)

Magnetic resonance imaging (MRI) is another noninvasive imaging tool that produces three-dimensional anatomic images similar to PET/CT without the need for ionizing radiation, which is ideal for applications that require frequent imaging, such as screenings for LN metastasis or recurrence. However, MRI is limited by low sensitivity (~35%), as traditional gadolinium contrast agents cannot differentiate healthy from metastatic LNs due to nonspecific accumulation. (11) Instead, MRI scans are used to visualize LN size and shape to determine metastasis. (12) However, these physical characteristics are poor diagnostic factors for LNs with micrometastases (<1 cm), resulting in misdiagnosis rates as high as 60%. (13,14) In addition, the safety of clinical gadolinium contrast agents has been of concern, as they have been demonstrated to accumulate in the brain, bones, and liver. (15)

Given these limitations, nanomedicine approaches have the potential to develop contrast agents with superior sensitivity and safety over gadolinium contrast agents. For example, peptide amphiphile micelles (PAMs) are nanoparticles self-assembled from amphiphilic monomers comprised of a bioactive peptide headgroup and a hydrophobic lipid tail. (16) In addition to peptides, the headgroup can be engineered to incorporate other payloads, such as gadolinium (Gd) chelates, to enable multimodal functionality, therapy, including imaging, and molecular targeting. (17) Furthermore, PAMs are synthesized using poly(ethylene glycol) (PEG) as a linker, which mitigates nanoparticle clearance by the mononuclear phagocyte system (MPS). (18) PAMs have also been demonstrated to be biocompatible and cleared within 7 days through renal filtration. (19) Given their multifunctionality, biocompatibility, and rapid clearance, PAMs represent an ideal nanoparticle carrier for the targeted delivery of MRI contrast agents to metastatic LNs.

Accordingly, there is a need for improved methods for imaging lymph node metastasis by magnetic resonance imaging.

### SUMMARY

In at least one aspect, an MRI contrast delivery system is provided, The MRI contrast delivery system includes nanoparticles that are each equipped with targeting peptides for CCR2 receptors and an MRI contrast moiety. These components are conjugated to the nanoparticles, which are made from hydrophobic lipids. This design enables the nanoparticles to “hitchhike” on monocytes, leveraging their natural migration pathways to efficiently target metastatic lymph nodes (LNs). This targeted delivery approach significantly enhances the specificity and sensitivity of the MRI signals, improving the imaging of metastatic sites and potentially aiding in more accurate diagnosis and assessment of cancer progression.

In another aspect, a diagnostic agent is designed for use in magnetic resonance imaging (MRI) to detect lymph node (LN) metastasis. This agent includes nanoparticles that incorporate gadolinium (Gd) and a peptide derived from the C—C chemokine receptor type 2 (CCR2)-binding motif of monocyte chemotactic protein 1 (MCP1). The configuration of the nanoparticles allows them to hitchhike on monocytes for targeted delivery to metastatic LNs, thereby enhancing the specificity and sensitivity of the MRI signal.

In another aspect, a gadolinium-loaded micelle platform targeted to tumor cells for the detection of lymph node metastases in cancer is provided. Specifically, micelles chelated with gadolinium ions and functionalized with a 23-residue peptide derived from the binding motif of the MCP1 protein (MCP1-Gd micelles) have been synthesized. These micelles target the C—C chemokine receptor 2 (CCR2), which is highly expressed across multiple cancers and also expressed in circulating monocytes known to migrate to lymph nodes affected by cancer [28]. Additionally, the interactions between MCP1-Gd micelles and monocytes are characterized in vitro, and the influence of circulating monocytes on MCP1-Gd accumulation in the lymph nodes will be investigated using in vivo models of metastatic melanoma.

In another aspect, techniques are provided for the accurate detection of cancer metastasis in lymph nodes which is crucial for patient treatment and prognosis. The standard clinical lymph node biopsy, while informative, often causes chronic pain and lymphedema while suffering from sampling bias. Conventional MRI does not provide the needed sensitivity and specificity for diagnosing metastases in normal-sized lymph nodes. To enhance MRI sensitivity, researchers developed MCP1-Gd micelles. These are chelated with gadolinium ions to enable MRI and functionalized with peptides targeting the CCR2 receptor, which is highly expressed in cancer. In vitro tests showed that MCP1-Gd micelles specifically bind to CCR2-expressing cells and produce a 55% higher T1-weighted MR signal in metastatic lymph nodes than in healthy ones. Further in vivo studies indicated that circulating CCR2+ monocytes help transport and accumulate these micelles in the lymph nodes, underscoring their potential as a contrast-enhancing tool in MRI for detecting lymph node metastases.

In another aspect, the techniques described herein relate to the MRI contrast delivery system wherein the nanoparticles are configured to hitchhike on monocytes for targeted delivery to metastatic lymph nodes, thereby enhancing MRI signal specificity and sensitivity.

In another aspect, the techniques described herein relate to a method for detecting lymph node (LN) metastasis in a subject using magnetic resonance imaging (MRI). The method involves: administering to the subject a composition that includes micelles incorporating gadolinium (Gd) and peptides derived from the CCR2-binding motif of MCP1; allowing for the migration of monocytes hitchhiked by the micelles to metastatic LNs; and conducting MRI to detect the enhanced signal from the metastatic LNs due to the accumulation of the gadolinium-incorporating micelles or fragments therefrom. Advantageously, the method enhances the sensitivity of MRI in detecting LN metastasis.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1. MCP1-Gd micelles accumulate in metastatic lymph nodes (MLNs) through CCR2-binding to cancer cells and monocyte hitchhiking.

FIGS. 2A, 2B, 2C, 2D, 2E, 2F, 2G, and 2H. Synthesis and characterization of MCP1-Gd micelles. (A) Scheme depicting the self-assembly of MCP1-Gd micelles. (B) TEM image confirming synthesis of MCP1-Gd micelles. Scale bar=50 nm. (C) Size (blue) and zeta potential (red) of MCP1-Gd micelles. N=3. (D) Phantom scans of MCP1-Gd micelles ranging from 0.0-1.0 mM Gd indicate an r1 relaxivity of 1.66 mM-1 s-1. (E) MCP1-Gd and scrMCP1-Gd are biocompatible with SVEC4-10 lymphatic endothelial cells and WEHI-274.1 monocytes after 48 h. N=6. In vitro micelle binding to (F) B16F10-Luc2 and (G) WEHI-274.1 cells after 1 h incubation demonstrate increased binding of MCP1-Gd. Data normalized to scrMCP1-Gd. Scale bar=50 μm. **p<0.01. ****p<0.001. N=6. (H) In vitro transwell assay evaluating the role of monocyte hitchhiking in MCP1-Gd transport across an endothelial barrier, indicating a 2-fold increase in micelle transport when incubated with migrating monocytes. Data normalized to micelle-only control. ***p<0.005. N=3.

FIGS. 3A, 3B, 3C, 3D, 3E, 3F, 3G, 3H, and 3I. In vivo lymph node accumulation of MCP1-Gd in a metastatic lymph node (MetLN) model. (A) Mice are inoculated with 200,000 B16F10-Luc2 cells and MRI scanned 14 days later. (B) Lymph node metastasis is confirmed through bioluminescence imaging of the ipsilateral inguinal lymph node 14 days after implantation of B16F10-Luc2 cells into the flanks of C57BL/6 mice. White arrows indicate the direction of lymphatic drainage. (C) Immunofluorescent staining of sectioned LNs indicates greater CCR2 expression (green) in metastatic LNs (MLNs) than control LNs (CLNs). Sections were counterstained with DAPI (blue). Scale bar=100 μm. (D) Flow cytometric analysis indicates greater CCR2 expression in MLNs than CLNs. *p<0.05. N=4. (E) Evaluation of CCR2 mRNA and cell surface expression through PCR (left) and flow cytometry (right) indicate an increase in CCR2 following LN metastasis. **p<0.01. ****p<0.001. N=6 for PCR and N=4 for flow cytometry. (F) Representative T1-weighted MRI scans of MetLN mice 3 h postinjection of MCP1-Gd or Magnevist. Purple and red boxes denote MLNs and CLNs, respectively. (G) Normalized MRI signal (top) and SNR (bottom) in MLNs and CLNs of MetLN mice i.v. injected with MCP1-Gd or Magnevist, indicating MLN targeting of MCP1-Gd. **p<0.01. ***p<0.005. N=6. (H) Normalized radiance measurements of LNs from MetLN mice indicate greater MCP1-Gd specificity for MLNs. ****p<0.001. N=8. (I) Quantification of short-axis diameters of LNs from MRI scans of mice containing LN metastases indicates no difference between MLNs and CLNs, confirming that LN size is a poor indicator of LN metastasis. N=12.

FIGS. 4A, 4B, 4C, 4D, and 4E. In vivo LN accumulation and distribution of MCP1-Gd in mice with metastatic LN recurrence (rMLN). (A) rMLN is established in 7 days after surgical resection of primary B16F10-Luc2 tumors and confirmed through bioluminescence imaging. (B) Representative MRI scan of C57BL/6 mouse with rMLN. Purple and red boxes denote rMLN and CLN, respectively. (C) Normalized MRI signal (left) and SNR (right) indicate rMLN targeting of MCP1-Gd 3 h after injection. *p<0.05. N=3. (D) Normalized radiance measurements from rMLNs and CLNs taken 3 h postinjection of MCP1-Gd confirm rMLN targeting. *p<0.05. N=3. (E) Immunofluorescent stains of sectioned rMLNs and CLNs indicate greater CCR2 expression (green) and MCP1-Gd accumulation (red) in rMLNs. Scale bar=250 μm.

FIGS. 5A, 5B, 5C, 5D, and 5E. Evaluation of the monocyte role in LN accumulation of MCP1-Gd. (A) Flow cytometry indicates increased number of CD11b+/CCR2+ monocytes in MLNs. **p<0.01. N=4. (B) Experimental timeline for in vivo monocyte depletion studies. (C) Cy7 radiance measurements from ex vivo images of LNs collected from monocyte-depleted or PBS-treated mice injected with MCP1-Gd or scrMCP1-Gd indicate LN accumulation of MCP1-Gd is reduced by 70% following monocyte depletion. Data normalized to PBS-treated mice. ***p<0.005. N=3. (D) Flow cytometric analysis of LNs from monocyte-depleted or PBS-treated mice indicate monocyte depletion reduces the percentage of LN cells bound to MCP1-Gd by >90%. ***p<0.005. N=3. (E) Immunofluorescent staining of LN sections from mice injected with MCP1-Gd or scrMCP1-Gd evaluating CCR2 (green), LYVE1 (blue), and micelle distribution (red). Scale bar=250 μm.

FIGS. 6A, 6B, 6C, 6D, and 6E. In vivo safety and biocompatibility of MCP1-Gd. (A) Timeline depicting experimental procedure for in vivo immunogenicity studies. (B) ELISA evaluating anti-PEG IgG and IgM indicates nonimmunogenicity of MCP1-Gd. N=4. (C) Plasma half-life of MCP1-Gd is unchanged between naïve mice and mice with prior exposure to MCP1-Gd following intravenous administration of micelles. N=4. (D) H&E stains of tissues collected from mice treated weekly with MCP1-Gd or PBS indicate no morphological damage associated with MCP1-Gd treatment. (I) Liver, (II) Kidney, (III) Spleen, (IV) Lung, (V) Heart, (VI) Intestine, (VII) Brain, (VIII) Lymph node. Scale bar=125 μm. (E) Evaluation of blood chemistry markers indicate no effect of weekly MCP1-Gd treatment on renal (BUN, creatinine) or hepatic (ALT, AST) health in mice relative to PBS-treated controls. N=4.

FIGS. 7A and 7B. Micelle size influences in vivo lymph node accumulation. A) Size measurements of micelles synthesized using different PEG lengths. *p<0.05. N=3. B) In vivo inguinal lymph node accumulation of different-sized Cy7-labeled micelles 3 h post-injection indicates that smaller micelle size trends toward favorable lymph node accumulation. *p<0.05. N=4.

FIG. 8. Transmission electron micrograph of scrMCP1-Gd confirms formation of spherical micelles ~12 nm in diameter. Scale bar=50 nm.

FIG. 9. R1 relaxivity of scrMCP1-Gd micelles (1.78 mM-1 s-1) is similar to that of the clinical contrast agent Magnevist (3.20 mM-1 s-1).

FIG. 10. In vitro micelle binding to NCI-H460 lung cancer cells confirms MCP1-Gd binding is dependent on CCR2 expression. Scale bar=50 μm. N=6.

FIG. 11. Translocation of FITC-dextran confirms the formation of an endothelial monolayer of SVEC4-10 cells. N=3.

FIG. 12. Transwell assay evaluating monocyte migration past a lymphatic endothelial monolayer confirms monocyte migration when stimulated with a chemoattractant (FBS). ****p<0.001. N=4.

FIG. 13. In vivo luciferase kinetic curve following i.p. injection into C57BL/6 mice. N=3.

FIG. 14. In vivo biodistribution of MCP1-Gd 3 h following tail-vein injection, indicating increased accumulation in MLNs relative to CLNs. ****p<0.001. N=8.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C6-10 aryl, C6-10 heteroaryl, —NO₂, —NH₂, —N(R′R″), —N(R′R″R′″) “L”, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C1-10 alkyl or C6-18 aryl groups; single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C6-10 aryl, C6-10 heteroaryl, —NO₂, —NH₂, —N(R′R″), —N(R′R″R′″)+L″, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C1-10 alkyl or C6-18 aryl groups; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

The term “conservative substitutions” describes the process of replacing one or more amino acids in a polypeptide with others that possess similar chemical and physical properties, thereby preserving the protein's structural integrity and functionality. Hydrophobic, nonpolar amino acids such as leucine, isoleucine, and valine are often exchanged for each other, with methionine also being a suitable substitute due to its hydrophobic nature. Similarly, the aromatic amino acids phenylalanine, tyrosine, and tryptophan can replace each other, although tyrosine is sometimes considered separately because of its polar hydroxyl group. The group of polar, uncharged amino acids includes serine, threonine, asparagine, glutamine, and occasionally cysteine, which is included because of its reactive thiol group. Among the basic amino acids, lysine, arginine, and histidine share similarities, though histidine is notable for its pH-sensitive properties. Acidic amino acids, such as aspartic acid and glutamic acid, are typically interchangeable. Special cases include glycine, recognized for its small size, and proline, known for its ring structure that influences protein folding; these are less commonly substituted but can be replaced with alanine or other small, non-critical amino acids in certain regions of the protein.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence and, in some embodiments, is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extends penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard (1)), BLAST2 (EMBL URL: http://www.embl-heidelberg.de/Services/index.html using default parameters: Matrix BLOSUM62; Filter: default, echofilter: on, Expect: 10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. In any peptide sequence provided herein, sequences with at least 70%, 80%, 90%, 95%, 98%, or 99% identity can be alternatively used.

The term “nanoparticle” refers to a particle having at least one dimension less than or equal to 100 nm.

Throughout this application, where publications are referenced, the disclosures of these publications in their entireties are hereby incorporated by reference into this application to more fully describe the state of the art to which this invention pertains.

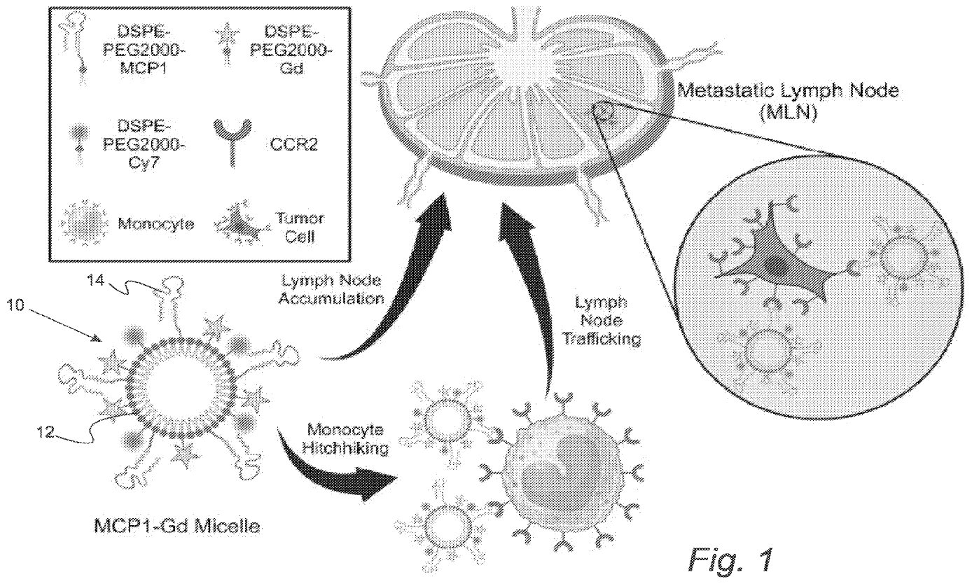

In at least one aspect, an MRI contrast delivery system for imaging metastasis and in particular, lymph node metastasis is provided. Referring to FIG. 1, the MRI contrast delivery system 10 includes a plurality of nanoparticles 12 wherein each nanoparticle includes one or more targeting peptides 14 that target the CCR2 receptor conjugated thereto and a magnetic resonance contrast moiety conjugated thereto. The magnetic resonance contrast moiety allows for the enhancement of magnetic resonance imaging. Characteristically, the nanoparticles are configured to hitchhike on monocytes for targeted delivery to metastatic LNs, thereby enhancing MRI signal specificity and sensitivity. Advantageously, the nanoparticles (e.g., micelles) demonstrate biocompatibility and lack of cytotoxicity in both in vitro and in vivo settings, making them safe for repeated use in diagnostic applications.

In another aspect, an MRI contrast delivery system for imaging metastasis in a subject, and in particular, for imaging lymph node metastasis in a subject is provided. The MRI contrast delivery system includes a plurality of nanoparticles wherein each nanoparticle includes CCR2 receptor-targeting peptides conjugated thereto and an MRI contrast moiety conjugated thereto. In a refinement, the plurality of nanoparticles includes nanoparticles selected from the group consisting of micelles, liposomes, lipid nanoparticles, and combinations thereof. In a refinement, the plurality of nanoparticles includes a plurality of single-layer micelles, also known as unilamellar micelles.

In another aspect, the nanoparticles have an average diameter of about 5 to 25 nm. In some refinement, the nanoparticles have an average diameter of at least 2, 3, 5, 8, or 10 nm and an average diameter of at most 50, 40, 30, 25, 20, or 15 nm.

In another aspect, the CCR2 receptor-targeting peptide is a fragment of a MCP1. In a refinement, the CCR2 receptor-targeting peptide is a fragment of a peptide having sequence QPDAINAPVTCCYNFTNRKISVQRLASYRRITSSKCPKEAVIFKTIVAKEICADPKQKWVQDS MDHLDKQTQTPKT (SEQ ID NO: 1). Typically such fragments include at least 10, 11, 12, 13, 14, 15, 16 or 17 contiguous amino acids from SEQ ID NO: 1 and at most 40, 35, 30, 28, 25, 23, 20 or 18 contiguous amino acids from SEQ ID NO: 1.

In another aspect, the CCR2 receptor-targeting peptide is a peptide having CYNFTNRKISVQRLASYRRITSSK (SEQ ID NO: 2) or a peptide thereof with 1 to 7 conservative substitutions. The peptide having SEQ ID NO: 2 is derived from human MCP1. In another refinement, the CCR2 receptor-targeting peptides include a fragment of SEQ ID NO: 2 having 5 to 18 contiguous amino acids or a peptide thereof with 1 to 7 conservative substitutions. In a refinement, the CCR2 receptor-targeting peptide is MCP1 with at least 70, 80, 90, 95, 98, or 99 percent identity to SEQ ID NO: 1). Examples of the peptide with conservated substitution include but are not limited to, CFNFTNRKISVQRLASYRRITSSK (SEQ ID NO: 3), CYNFTNRRISVQRLASYRRITTSK (SEQ ID NO: 4), CYNFTNRRISVQRLAAYRRITTSK (SEQ ID NO: 5), CYQFTNRKISVQRLASYRRITSSK (SEQ ID NO: 6), CYNFTNRKLSVQRLASYRRILSSK (SEQ ID NO: 7), or CFNFTNRRISVQRLAFYRRITTSK (SEQ ID NO: 8).

Advantageously, the CCR2 receptor-targeting peptides and the MRI contrast moiety are independently connected to the nanoparticles by reaction with a functional group. For example, the functional group that can be used for linking includes amines, carboxylic acids, NHS esters, acid anhydrides, or unsaturated imides (e.g., maleimide). In a refinement, the functional group for the MRI contrast moiety includes a chelating moiety. The chemistry described herein is related to the functionalization of nanoparticles, particularly for attaching targeting peptides and MRI contrast moieties through various types of linkages. This functionalization is key in the design of targeted imaging agents, where both specificity and imaging contrast are crucial. Various methods are employed for attaching molecules, such as peptides, to nanoparticles, which is crucial for developing sophisticated diagnostic tools. One common method involves forming robust amide bonds between amines and carboxylic acids by activating the acid with EDC (1-ethyl-3-(3-dimethylaminopropyl) carbodiimide, which creates a reactive O-acylisourea intermediate that couples with an amine. In another example, NHS esters also form amide bonds with amines under mild conditions and are particularly efficient for labeling peptides with functional groups such as dyes, drugs, or contrast agents, reacting specifically with primary amines. Another method utilizes acid anhydrides, which can react with amines to produce one or two amide bonds, introducing carboxylic acid functionalities that can be further modified. Additionally, maleimide groups are used for coupling with thiols, forming stable thioether bonds specifically with cysteine residues in peptides, a process that preserves the biological activity of peptides. Overall, the choice of linking chemistry depends on the functionality present on both the nanoparticle and the molecule to be attached (peptide or contrast agent). These methods allow for the precise and stable attachment of bioactive molecules and imaging agents to nanoparticles, enabling the development of sophisticated diagnostic tools.

In another aspect, chelating moieties are derived from chelators like DOTA (1,4,7,10-tetraazacyclododecane-N,N′,N″,N′-tetraacetic acid) and DTPA (diethylenetriaminepentaacetic acid), which are used to securely hold metal ions such as gadolinium, attaching to nanoparticles through any of the aforementioned methods depending on the functional groups involved.

In another aspect, the MRI contrast moiety includes gadolinium, and in particular, gadolinium cations (e.g., Gd(III) or Gd³⁺). In another variation, the MRI contrast moiety includes iron oxide. In still another variation, the MRI contrast moiety includes manganese, and in particular, manganese cations (e.g., Mn(II) or Mn²⁺).

As set forth above, the plurality of nanoparticles can include a plurality of micelles. In this regard, each micelle can include a plurality of targeting peptide-conjugated amphiphiles, a plurality of MRI contrast moiety-conjugated amphiphiles, and an optional plurality of non-targeted amphiphiles. In a refinement, the plurality of targeting peptide-conjugated amphiphiles includes amphiphiles having a first phospholipid conjugated to a CCR2 receptor-targeting peptide with a first linking group. In a refinement, the plurality of contrast moiety-conjugated amphiphiles includes amphiphiles having a second phospholipid conjugated to an MRI contrast moiety with a second linking group, and the optional plurality of non-targeted amphiphiles includes amphiphiles having a third phospholipid conjugated to a capping moiety with a third linking group. In a further refinement, the first linking group, the second linking group, and the third linking group are each independently a polyethylene glycol having a weight average molecular weight from about 500 to 10000 Daltons. In a further refinement, the first phospholipid, the second phospholipid, and the third phospholipid are each independently selected from the group consisting of phosphatidic acids, phosphatidyl inositols, phosphatidyl cholines, phosphatidyl ethanolamines, phosphatidyl serines, phosphatidyl glycerols, and any combinations thereof. In some examples, the first phospholipid, the second phospholipid, and the third phospholipid are each independently selected from the group consisting of phosphatidylglycerol, lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine, palmitoyloleoylphosphatidylcholine, di stearoylphosphatidylcholine, dioleoylphosphatidylcholine, dipalmitoylphosphatidylcholine, dioleoylphosphatidylglycerol, dipalmitoylphosphatidylglycerol, -phosphatidylethanolamine, dioleoyl-phosphatidylethanolamine 4-(N-maleimidomethyl)-cyclohexane-1-carboxylate (DOPE-mal), 1-stearoyl-2-oleoyl phosphatidylcholine, 1,2-distearoyl-sn-glycerol-3-phosphoethanolamine, and combinations thereof.

In another aspect, the MRI contrast delivery system further includes a pharmaceutically acceptable carrier (e.g., saline). This carrier serves multiple purposes: it acts as a solvent or diluent for the MRI contrast agents, ensuring they are in a suitable form for administration, and it aids in the dispersion and stabilization of the nanoparticles or other contrast media throughout the administration process. Saline, being isotonic with body fluids, is particularly favored as it minimizes irritation and is compatible with biological tissues, making it an ideal choice for intravenous injections. Furthermore, the use of a pharmaceutically acceptable carrier like saline is crucial for maintaining the bioavailability and optimal pharmacokinetic properties of the contrast agents, ensuring they reach their target tissues or organs effectively without precipitating or degrading prematurely. This careful formulation enhances the diagnostic capabilities of the MRI procedure by providing clearer, more accurate imaging results while maintaining patient safety and comfort.

In another aspect, a chemotherapeutic agent is conjugated to the nanoparticles or encapsulated by the nanoparticles. This strategy significantly enhances the delivery mechanisms of cancer treatments. When a chemotherapeutic agent is conjugated to nanoparticles, it is typically covalently bonded to the surface or the matrix of the nanoparticles. This approach allows for targeted delivery, where the chemotherapeutic can be released in a controlled manner directly at the tumor site, reducing the overall systemic toxicity and increasing the drug's efficacy against cancer cells. Alternatively, encapsulating the chemotherapeutic agent within the nanoparticles offers another sophisticated method of drug delivery. In this setup, the drug is physically enclosed within the nanoparticle, which may be composed of lipids, polymers, or other suitable materials that can carry the drug molecules. Encapsulation protects the chemotherapeutic agent from the external environment, potentially enhancing its stability and solubility. Moreover, it can provide a sustained release profile, where the drug is gradually released over time, maintaining therapeutic concentrations in the target area for longer periods. Both conjugation and encapsulation methods aim to exploit the unique properties of nanoparticles, such as their small size, large surface area to volume ratio, and the ability to modify their surface with specific ligands. These features can be harnessed to increase the drug's accumulation at the specific site of action while minimizing off-target effects and adverse reactions. This targeted approach not only improves the effectiveness of the treatment but also enhances patient outcomes by limiting the harsh side effects often associated with conventional chemotherapy treatments.

In another aspect, a method for imaging lymph node metastasis in a subject is provided. The method includes administering the MRI contrast delivery system described herein to a subject at risk for having lymph node metastasis. MRI images are taken of the subject to determine the presence of lymph node metastasis. In a refinement, the lymph node metastasis includes micro metastasis.

In some aspects, the techniques described herein relate to a method for detecting lymph node (LN) metastasis in a subject using magnetic resonance imaging (MRI). The method includes a step of administering to the subject a composition including micelles incorporating gadolinium (Gd) and peptides derived from the CCR2-binding motif of MCP1 and then allowing for the migration of monocytes hitchhiked by the micelles to metastatic LNs. MRI is then conducted to detect the enhanced signal from the metastatic LNs due to the accumulation of the gadolinium-incorporating micelles or fragments therefrom.

In another aspect, the method includes a detecting step that quantifies the MRI signal enhancement in the lymph nodes (LNs), thereby providing a measure of metastatic burden.

In another aspect, the method includes a step of peptide synthesis using solid-phase peptide synthesis techniques.

In another aspect, the method further includes a step of purifying the peptide using high-performance liquid chromatography (HPLC).

In another aspect, the method further includes a step of conjugation of the peptide to the lipid tail using a maleimide-functionalized lipid.

In another aspect, the method further includes a step of characterizing the micelles using dynamic light scattering to determine their size.

In another aspect, a method for manufacturing the diagnostic agent set forth above is provided. The method includes synthesizing the peptide derived from the MCP1 CCR2-binding motif, conjugating the peptide to nanoparticles incorporating gadolinium, and verifying the capability of the resultant micelles to hitchhike on monocytes.

Additional details are found in Trac N, Chen Z, Oh H S, Jones L, Huang Y, Giblin J, Gross M, Sta Maria N S, Jacobs R E, Chung E J. MRI Detection of Lymph Node Metastasis through Molecular Targeting of C—C Chemokine Receptor Type 2 and Monocyte Hitchhiking. ACS Nano. 2024 Jan. 23; 18(3):2091-2104. doi: 10.1021/acsnano.3c09201. Epub 2024 Jan. 11. PMID: 38212302 and related supplemental information, the entire disclosure of which is incorporated by reference in their entity.

The following examples illustrate the various embodiments of the present invention. Those skilled in the art will recognize many variations that are within the spirit of the present invention and scope of the claims.

To target metastatic lymph nodes (LNs), C—C chemokine receptor 2 (CCR2) was selected as a molecular target because it is expressed in most cancers and is further upregulated in metastatic LNs. (20) This upregulation is attributed to the pro-tumoral functions of CCR2 signaling, which include the secretion of angiogenic factors, the expression of proteases that promote metastasis, and the suppression of adaptive immunity. (21) Additionally, metastatic cancer cells secrete monocyte chemotactic protein 1 (MCP1), the natural ligand of CCR2. (22) MCP1 binds to CCR2 expressed by inflammatory monocytes, leading to chemotactic migration to the metastatic LN. (23) The inherent migration of monocytes in response to MCP1 has been reported as an effective strategy for targeted drug delivery. (24) For instance, Kuang et al. developed liposomes that hitchhiked onto monocytes through CD14 binding, resulting in enhanced delivery of doxorubicin to glioblastomas in vivo. (25) Therefore, nanoparticle hitchhiking onto inflammatory monocytes through molecular targeting of CCR2 may offer an additional mechanism for the targeted delivery of MRI contrast agents to metastatic LNs.

In the experiments below, PAMs functionalized with the CCR2-binding motif of MCP1 were synthesized to target metastatic lymph nodes (LNs) and incorporated gadolinium (Gd) to enable MRI detection (MCP1-Gd). (26) It was hypothesized that MCP1-Gd would detect LN metastasis earlier than clinical MRI through a combination of metastatic cancer cell targeting and monocyte hitchhiking (FIG. 1). To test this hypothesis, MCP1-Gd was synthesized and its in vitro biocompatibility, binding, and hitchhiking onto monocytes were confirmed. When injected into in vivo models of LN metastasis and LN recurrence, MCP1-Gd targeted metastatic LNs and enabled MRI detection earlier than with a clinical gadolinium contrast agent standard. Collectively, the studies demonstrate the potential of molecular CCR2 targeting for early detection of LN metastasis.

### Results & Discussion

### Synthesis and Characterization of MCP1-Gd

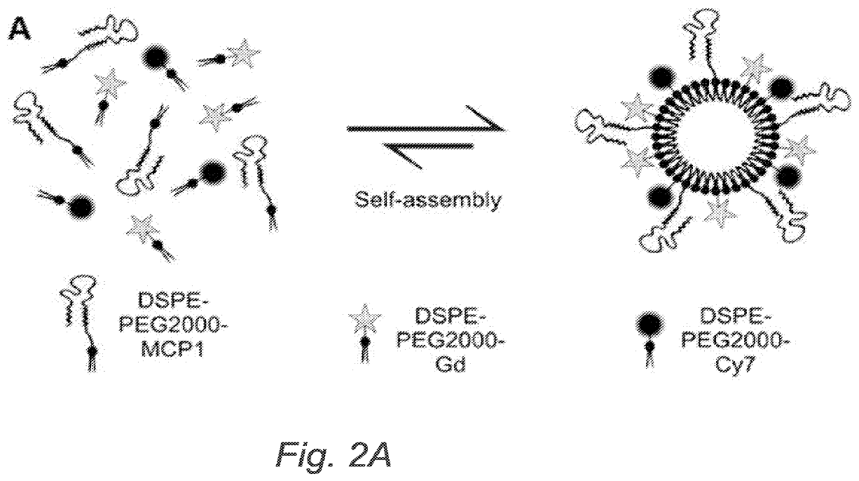

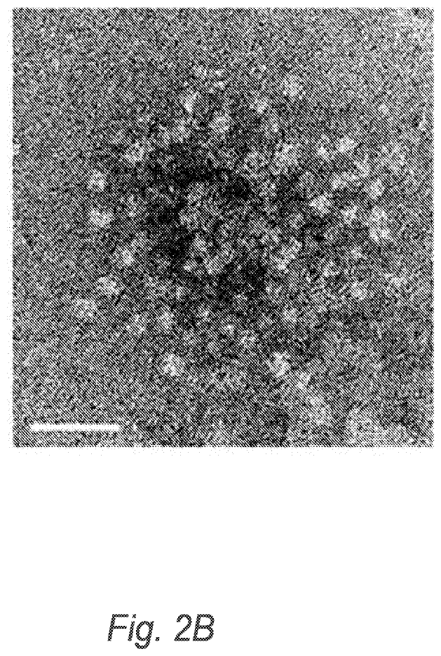

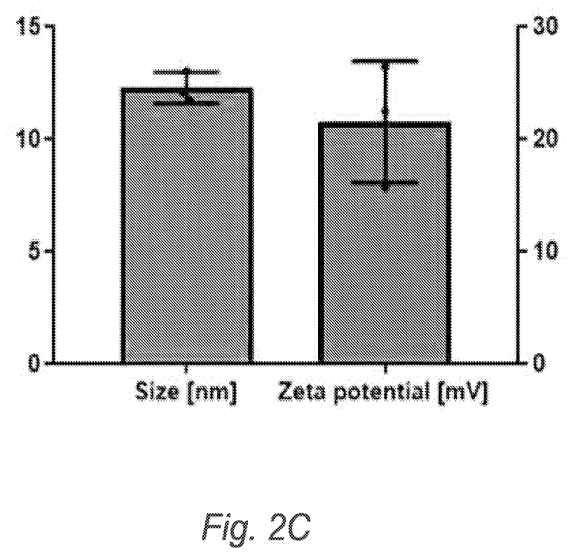

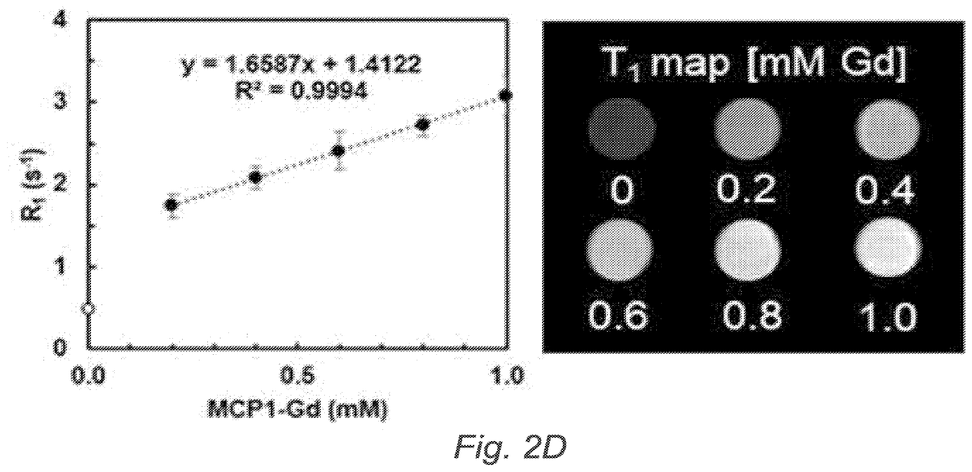

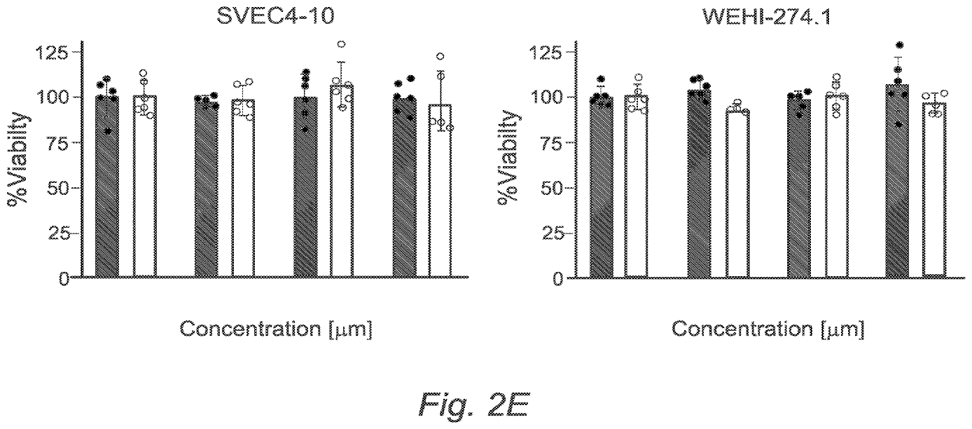

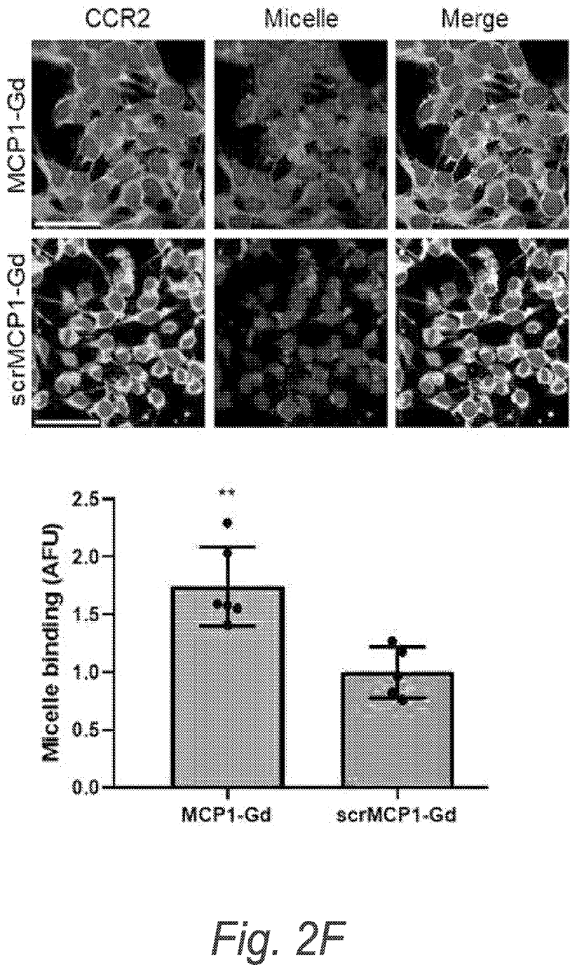

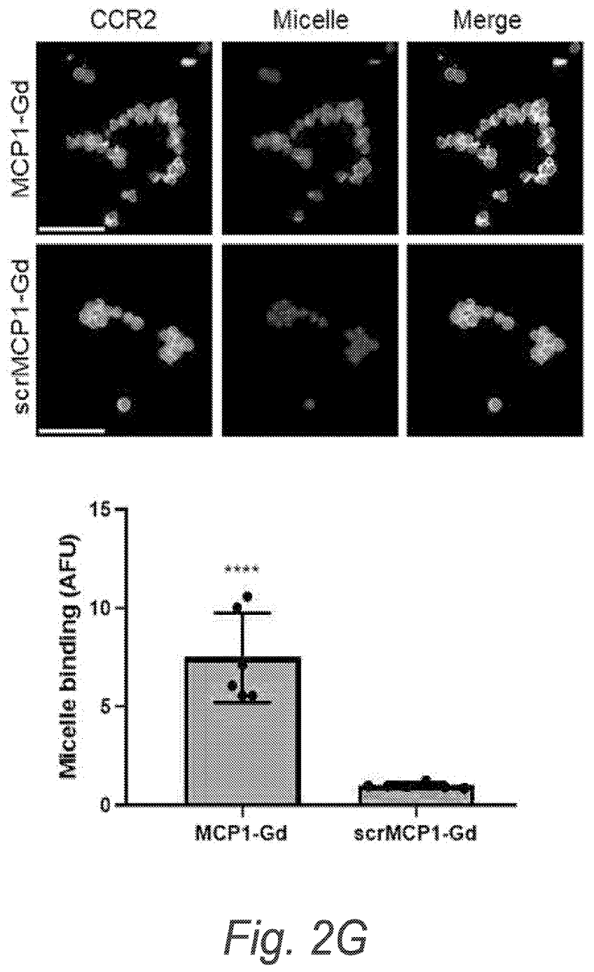

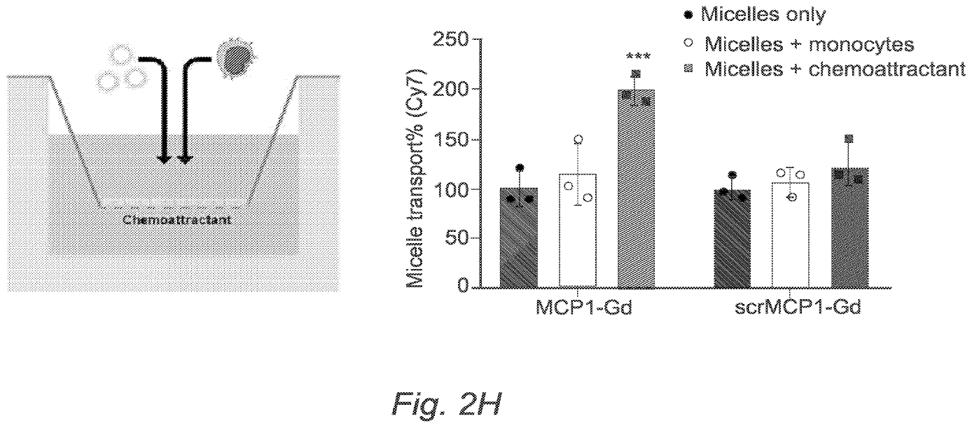

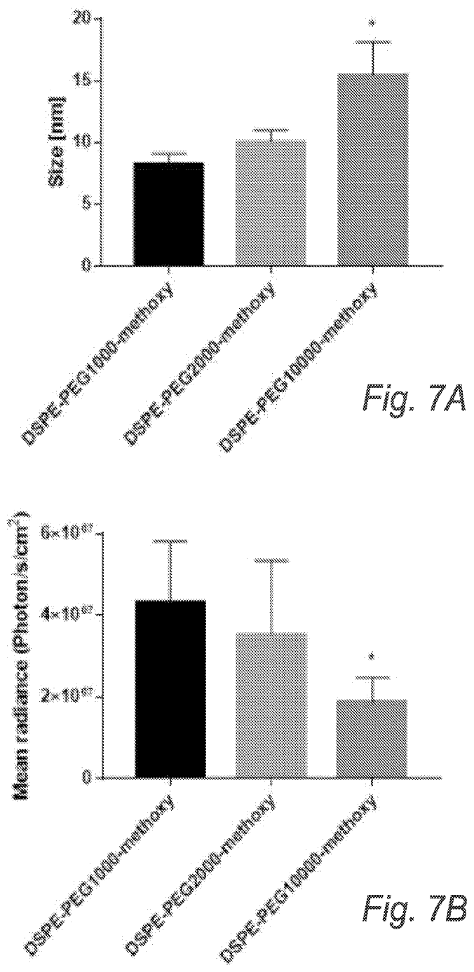

Because lymph nodes (LNs) have been reported to have size-exclusion barriers similar to those observed in the kidneys, a pilot study was first conducted to determine the optimal micelle size for LN targeting. (27-30) Cy7-labeled 1,2-distearoyl-sn-glycero-3-phosphoethanolamine (DSPE)-PEG-methoxy micelles of different sizes, ranging from 8.3 to 15.5 nm, were synthesized by varying the PEG molecular weight from 1000 to 10000 Da (FIG. 7A). Ex vivo fluorescence imaging of inguinal LNs collected 3 hours following tail-vein injection indicated that smaller micelles had greater LN accumulation, with a significant decrease in accumulation in the larger DSPE-PEG10000 micelles, suggesting the presence of a size cutoff around the 15 nm range for the amphiphilic micelles (FIG. 7B). No significant differences were observed in the micelle size or LN accumulation between DSPE-PEG1000 and DSPE-PEG2000 micelles. Thus, DSPE-PEG2000 was selected for all further studies due to its higher yield during synthesis. Monocyte chemotactic protein 1 (MCP1) is a chemokine whose primary function is to bind to circulating monocytes through the C—C chemokine receptor 2 (CCR2) and induce monocyte migration toward sites of inflammation, such as metastatic LNs. (31) To enable specific binding, a 23-residue peptide derived from the CCR2-binding motif of MCP1 (MCP1 peptide), as well as a scrambled peptide control (scrMCP1), were synthesis using solid-phase peptide synthesis. (32) Peptides were then purified through high-performance liquid chromatography (HPLC) and characterized through matrix-assisted laser desorption/ionization time-of-flight mass spectrometry (MALDI-TOF-MS). After purification, peptides were conjugated to DSPE-PEG2000-maleimide through thioether linkage to form peptide amphiphiles. Gadolinium (Gd) and Cy7 amphiphiles were similarly synthesized, purified, and characterized following conjugation to DSPE-PEG2000-amine through peptide bonds. (33) MCP1-Gd and control micelles were synthesized through thin-film hydration (FIG. 2A), and micelle formulations are given in Table 1.

TABLE 1
Micelle formulations

| Micelle Name | Micelle formulation |
|---|---|
| MCP1-Gd | 45% MCP1, 45% Gd, 10% methoxy |
| scrMCP1-Gd | 45% scrMCP1, 45% Gd, 10% methoxy |
| Cy7-labeled MCP1-Gd | 45% MCP1, 45% Gd, 10% Cy7 |
| Cy7-labeled scrMCP1-Gd | 45% scrMCP1, 45% Gd, 10% Cy7 |
| FITC-labeled scrMCP1-Gd | 45% MCP1, 45% Gd, 10% FITC |
| FITC-labeled scrMCP1-Gd | 45% scrMCP1, 45% Gd, 10% FITC |

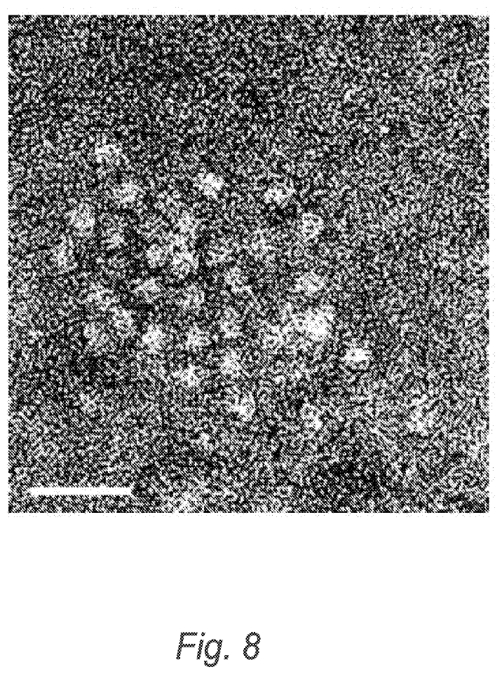

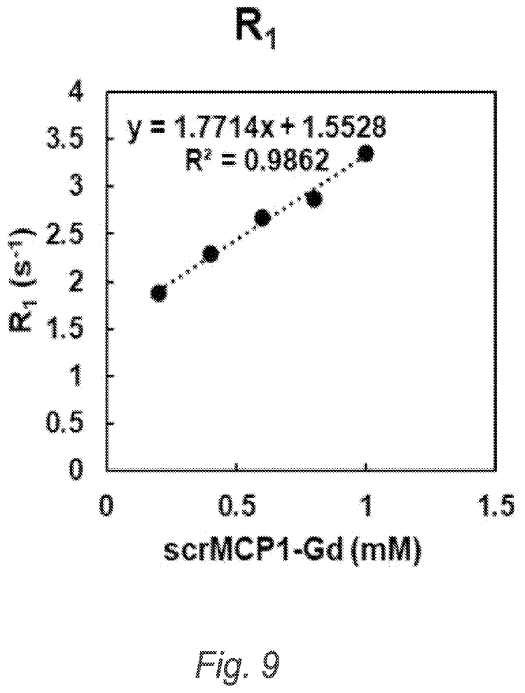

Transmission electron microscopy (TEM) indicated MCP1-Gd micelles were spherical with a mean diameter of 12.3 nm, which was corroborated by dynamic light scattering (DLS). To characterize the feasibility of MCP1-Gd as a diagnostic tool for MRI, a 7T PET-MR system was used to determine the r1 relaxivity of MCP1-Gd as 1.66 mM-1 s-1, which is comparable to the relaxivity of the clinical contrast agent Magnevist (Gd-DTPA) (3.20 mM-1 s-1) (FIG. 2D). (34) In addition, micelles incorporating a scrambled MCP1 peptide (scrMCP1-Gd) were also synthesized and found to have similar diameters and r1 relaxivities as MCP1-Gd (12.6 nm and 1.78 mM-1 s-1, respectively, FIGS. 8 & 9).

### In Vitro MCP1-Gd Biocompatibility, Binding, and Hitchhiking

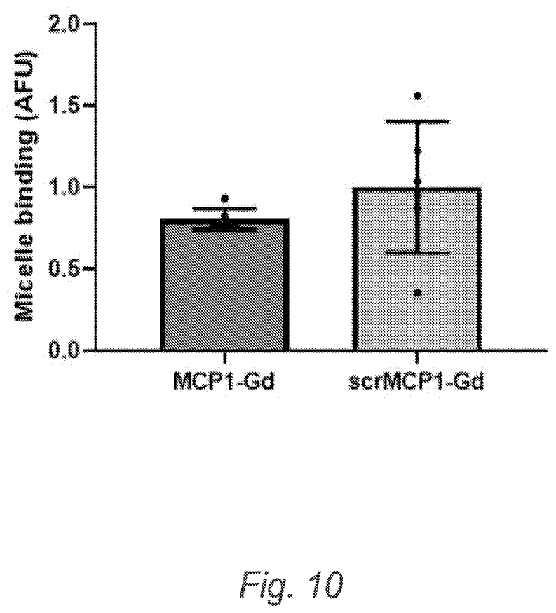

Because cationic surface charges have been reported to induce cytotoxicity in the literature through disruption of Na+, K+, and Ca2+ homeostasis, the in vitro biocompatibility of MCP1-Gd was characterized by incubating SVEC4-10 lymphatic endothelial cells or WEHI-274.1 monocytes with MCP1-Gd or scrMCP1-Gd for 48 hours, during which no cytotoxicity was observed (>90% viability) (FIG. 2E). (35,36) Following confirmation of biocompatibility, CCR2-expressing B16F10-Luc2 melanoma cells and WEHI-274.1 monocytes were incubated with MCP1-Gd or scrMCP1-Gd in vitro to verify CCR2 binding and specificity. B16F10-Luc2 cells treated with MCP1-Gd exhibited 50% more Cy7 fluorescence than cells treated with scrMCP1-Gd, indicating increased targeting of MCP1-Gd (FIG. 2F, p<0.01). Similarly, MCP1-Gd displayed 7-fold more binding to WEHI-274.1 monocytes than scrMCP1-Gd (FIG. 2G, p<0.001). As a control, the binding assay was repeated using NCI-H460 cells, a CCR2-lung cancer cell line, and no differences in binding were observed, confirming that MCP1-Gd binding is dependent on CCR2 expression (FIG. 10). (37)

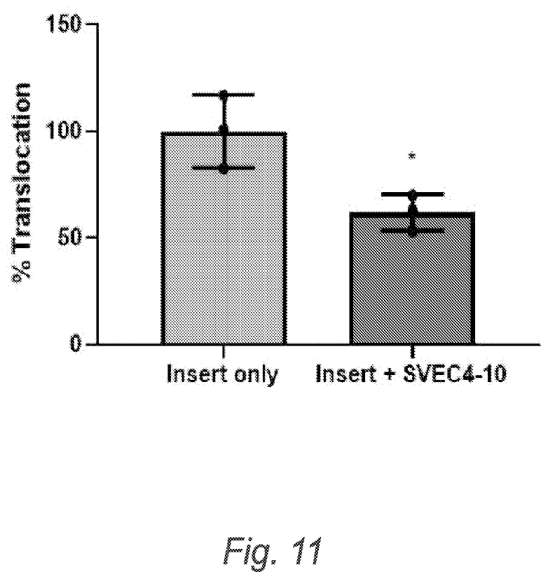

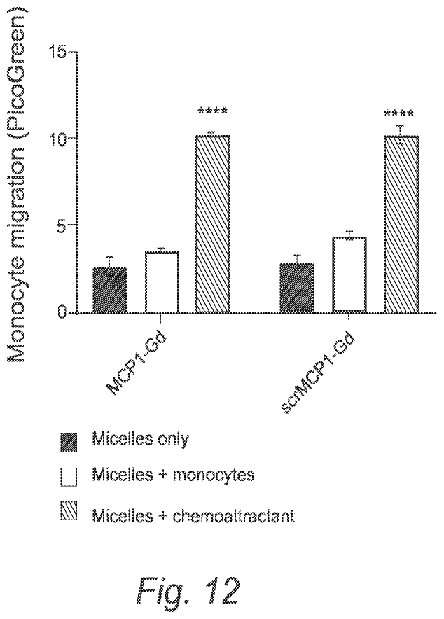

Recent studies have reported that molecular targeting can enable nanoparticle hitchhiking onto monocytes, which facilitates transport across endothelial barriers and thus enhances delivery to target tissues. (38) To assess the effect of monocyte hitchhiking on MCP1-Gd transport, an in vitro model of the lymphatic endothelium was developed by seeding SVEC4-10 cells into a transwell insert (FIG. 11). After the cells were allowed to form a monolayer, MCP1-Gd transport across the endothelial barrier were evaluated with or without migrating monocytes. DNA quantification through the PicoGreen assay confirmed monocyte migration in response to chemoattractant and also demonstrated that MCP1-Gd binding did not inhibit monocyte migration (FIG. 12). In addition, incubating MCP1-Gd with migrating monocytes increased micelle transport 2-fold compared to micelle-only controls (FIG. 2H, p<0.005). Importantly, there was no change in transport when micelles were incubated with nonmigrating monocytes (no chemoattractant). In addition, no changes in micelle transport were observed when scrMCP1-Gd was incubated with monocytes, indicating that monocyte hitchhiking is facilitated by CCR2-targeting and improves the transport of MCP1-Gd across endothelial barriers in vitro, representing an additional mechanism for metastatic LN targeting.

### In Vivo Targeting of MCP1-Gd in a Metastatic Lymph Node (MetLN) Model

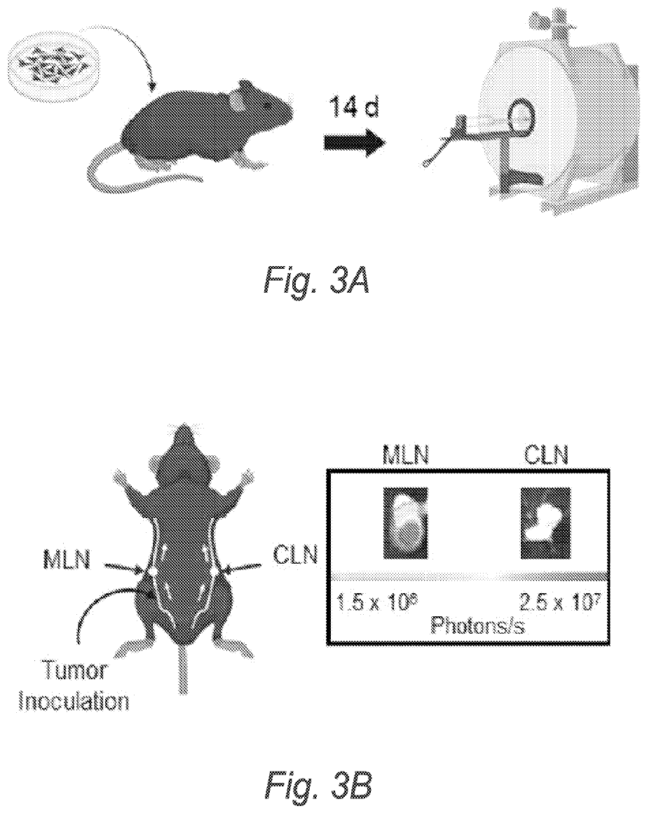

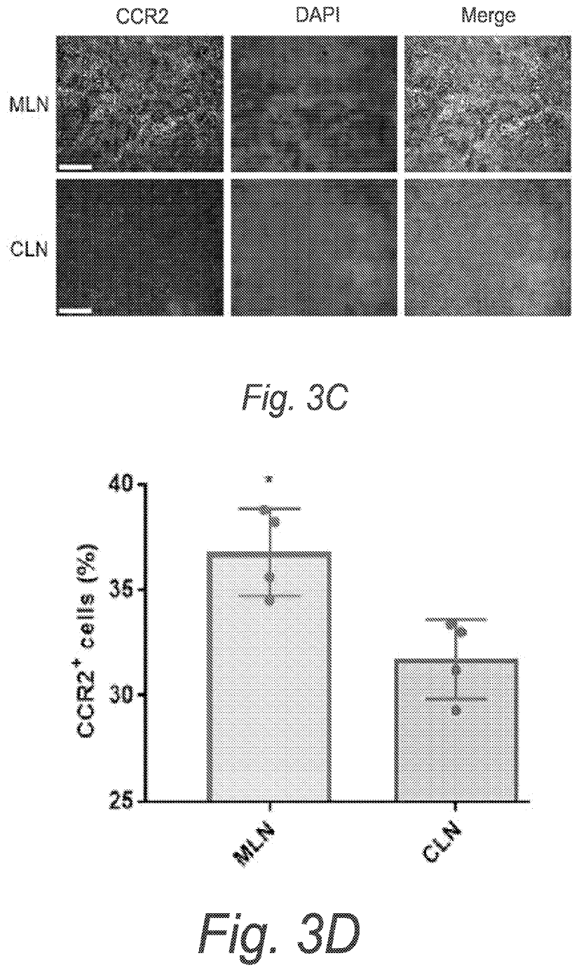

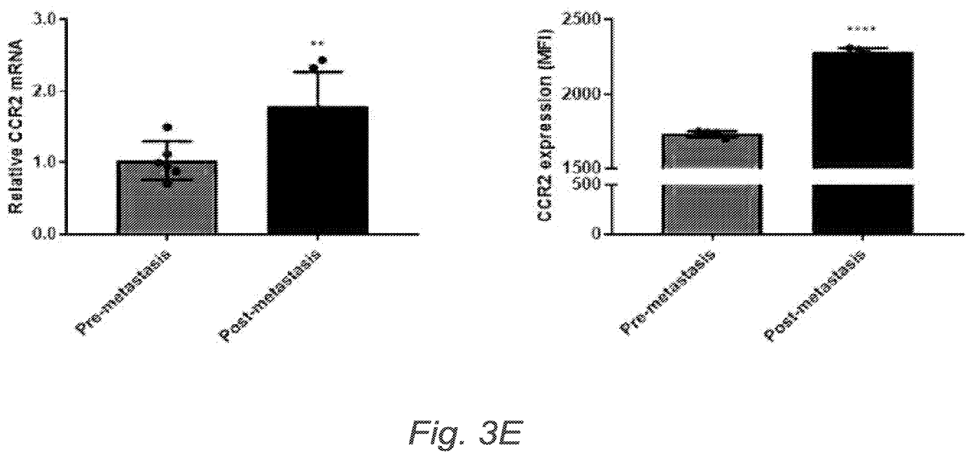

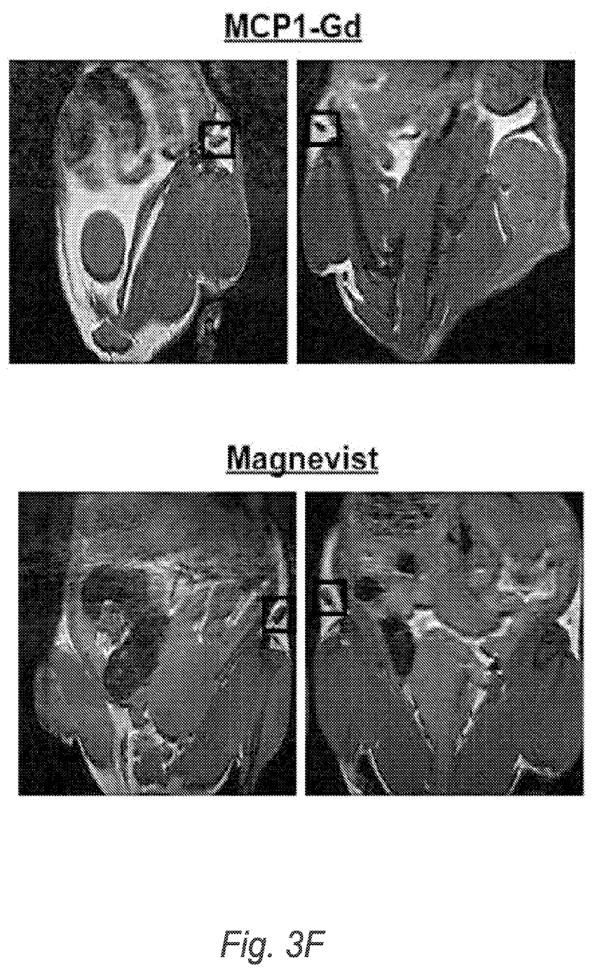

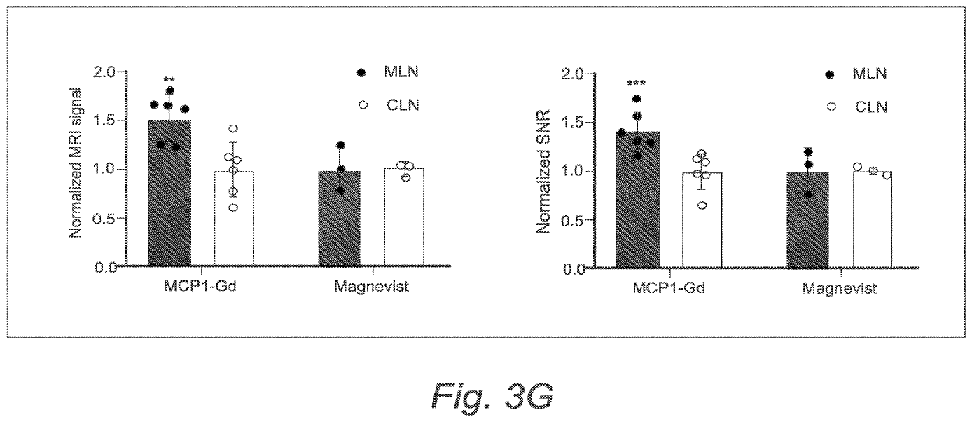

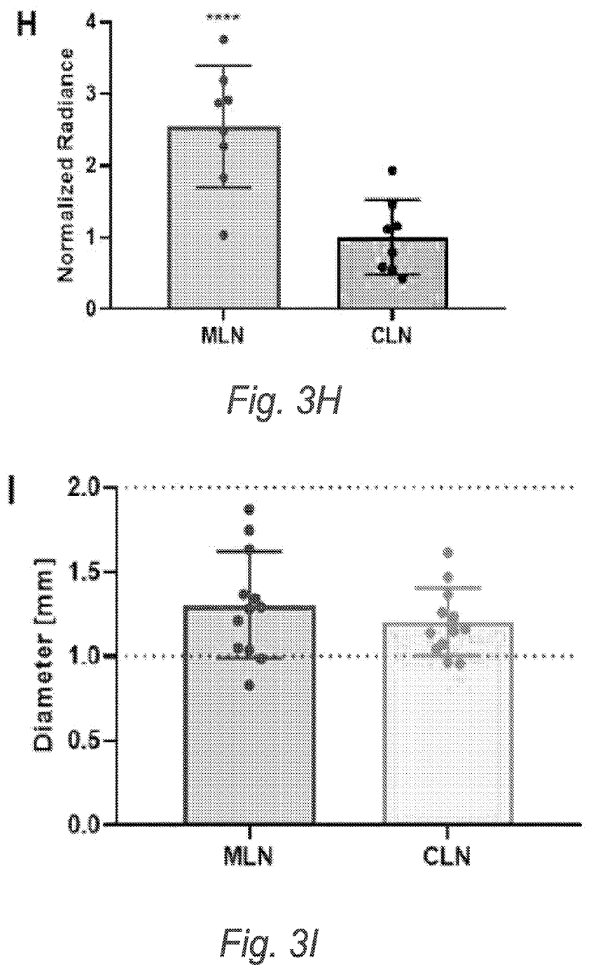

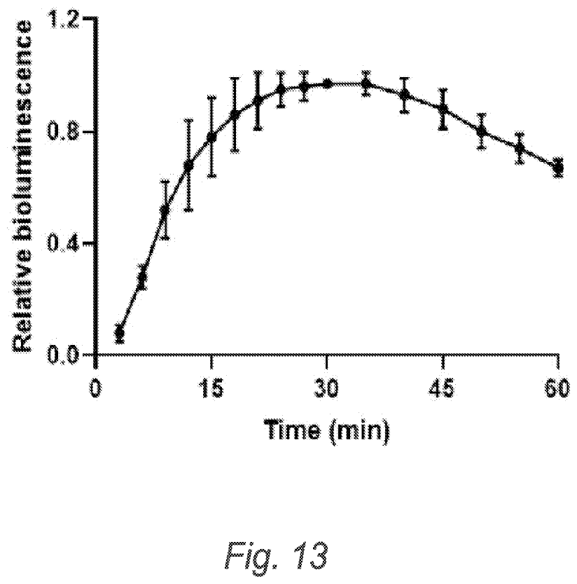

After confirming that monocyte hitchhiking improved the transport of MCP1-Gd across a lymphatic barrier in vitro, MCP1-Gd targeting of metastatic LNs was investigated in vivo (FIG. 3A). First, a metastatic lymph node (MetLN) mouse model was developed by implanting luciferase-expressing B16F10-Luc2 cells into the flanks of C57BL/6 mice (FIG. 13). After 14 days, bioluminescence imaging confirmed the presence of metastasis in the ipsilateral inguinal LN (MLN), while no metastasis was detected in the contralateral inguinal LN (CLN, FIG. 3B). After confirming the development of the in vivo MetLN model, CCR2 expression in the LNs of MetLN mice were characterized. Immunofluorescent staining of sectioned LNs indicated increased CCR2 expression in MLNs compared to that in CLNs (FIG. 3C). In addition, flow cytometry indicated that MLNs contained more CCR2+ cells, confirming the feasibility of CCR2 as an in vivo biomarker for MLNs (FIG. 3D, p<0.05). To further determine the viability of CCR2 as a molecular target for LN metastasis, B16F10-Luc2 cells were isolated from metastatic LNs and used polymerase chain reaction (PCR) and flow cytometry to evaluate the CCR2 expression (FIG. 3E). In agreement with literature reports correlating CCR2 with cancer progression and metastasis, increases in both mRNA and protein levels of CCR2 following LN metastasis (p<0.01) were observed. (39-41) These characterization studies indicate that CCR2 is highly expressed in MLNs and thus is a feasible biomarker for MLN targeting in vivo.

After verifying increased CCR2 expression in MLNs, MCP1-Gd were injected into MetLN mice and performed MRI scans 3 h after injection to evaluate MLN detection (FIG. 3F). The T1-weighted signal and signal-to-noise ratio (SNR) were 50% higher in MLNs compared to CLNs, confirming MLN detection through molecular targeting of CCR2 (FIG. 3G, p<0.01). Furthermore, no differences in signal nor SNR between LNs of mice administered with the clinical contrast agent Magnevist were observed thereby demonstrating the advantages of MLN targeting for MRI. Ex vivo imaging of LNs corroborated the findings from the MRI scans, with the Cy7 signal in the MLNs approximately 2.5× greater than the CLNs following MCP1-Gd injection (FIGS. 3H, p<0.001). Lastly, ImageJ was used to characterize LN size, as LN enlargement is used clinically to identify LN metastasis via MRI. (42,43) As shown in FIG. 3I, MLN size was not altered relative to CLNs (1.31 vs 1.20 mm, p=0.358). Moreover, none of the LNs measured were larger than 2 mm, the reported upper limit for normal LN size in C57BL/6 mice, reflecting literature reports indicating the poor diagnostic power of LN size in MRI and highlighting the need for improved diagnostic tools for identifying LN metastasis. (44,45) These studies demonstrate that through molecular targeting of MLNs, MCP1-Gd enables MRI detection of LN metastasis earlier than that of clinical contrast agents.

In Vivo Targeting of MCP1-Gd in a Metastatic LN Recurrence (rMLN) Model

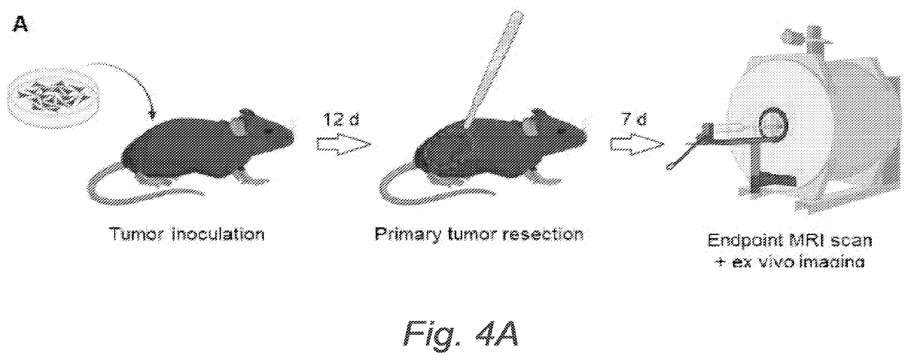

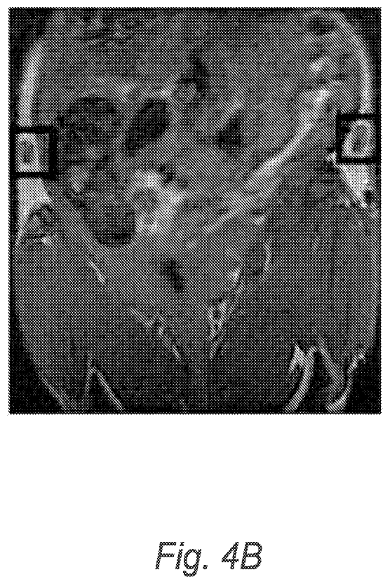

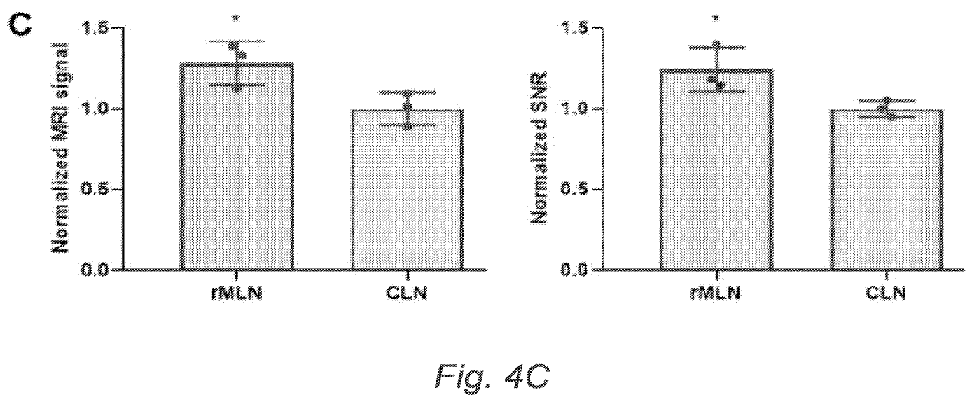

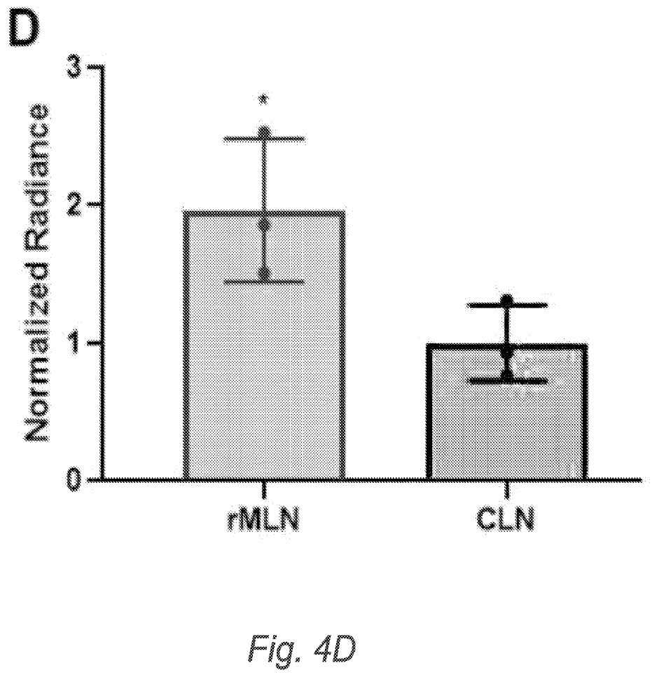

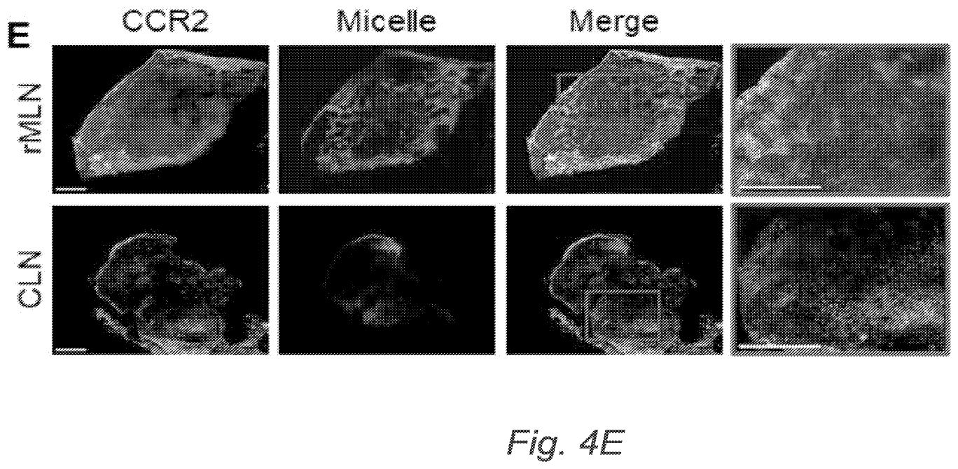

The early detection of melanoma recurrence identifies patients for adjuvant chemo- and immunotherapies, which has been reported to more than double patient survival rates. (46) Since the LNs are the most common site of recurrence in melanoma patients, the potential of MCP1-Gd was tested as a follow-up diagnostic tool for evaluating recurrent LN metastasis following surgical resection of a primary tumor. (47,48) An in vivo model of metastatic LN recurrence (rMLN) was developed by implanting B16F10-Luc2 cells into the flanks of C57BL/6 mice and resecting the tumors after 12 days. The presence of rMLNs was confirmed through bioluminescence imaging 7 days following surgery. Then, as shown in FIG. 4A, MCP1-Gd were injected into rMLN mice and performed MRI scans, which indicated 30% higher signal intensity and SNR in rMLNs relative to CLNs, confirming detection of metastasis (FIGS. 4B,C, p<0.05). Ex vivo fluorescence imaging also demonstrated greater MCP1-Gd signal in the rMLNs, approximately 2-fold that of the CLNs (FIGS. 4D, p<0.05). In addition, confocal microscopy images indicated that MCP1-Gd was distributed throughout the rMLN, while micelles in the CLN were limited to the outer capsule (FIG. 4E). This difference in distribution could be due to increased monocyte hitchhiking to the rMLNs, as monocytes enter the LNs via high endothelial venules (HEVs) located within the inner cortex. (49) Further immunofluorescent staining of rMLN sections for the cancer marker Ki67 and monocyte marker CD11b indicated that micelle signal colocalized more closely with monocytes than with metastatic cancer cells (Pearson's Correlation Coefficient: 0.28 vs 0.08). Collectively, these studies demonstrate the potential of MCP1-Gd as a follow-up diagnostic tool for the identification of metastatic recurrence in the LNs.

Evaluation of MCP1-Gd Hitchhiking onto Monocytes In Vivo

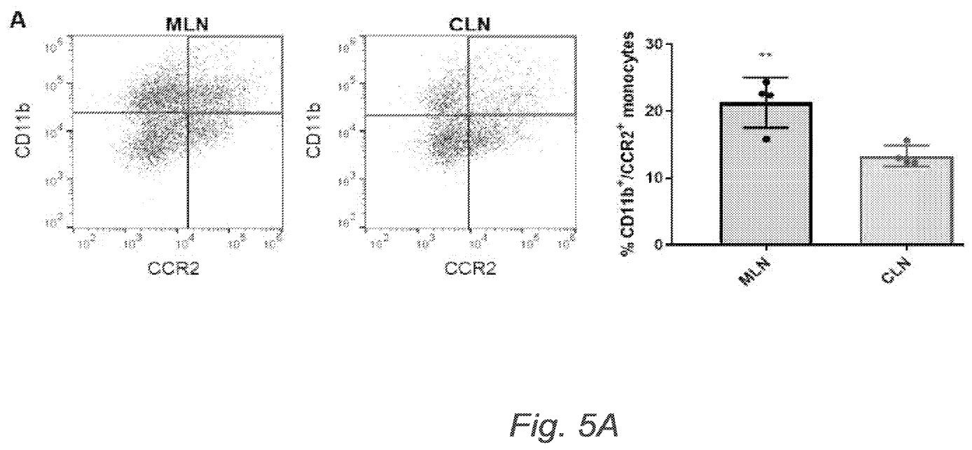

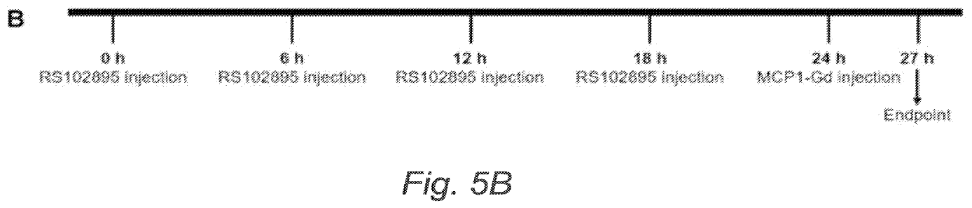

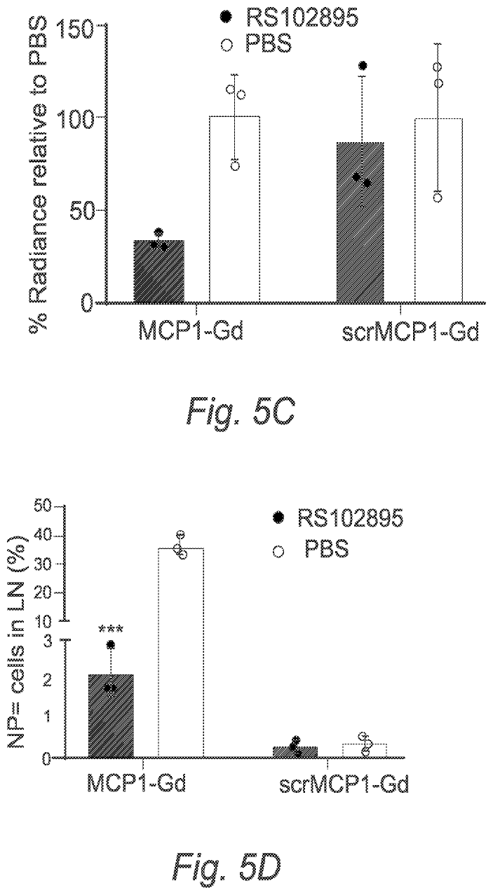

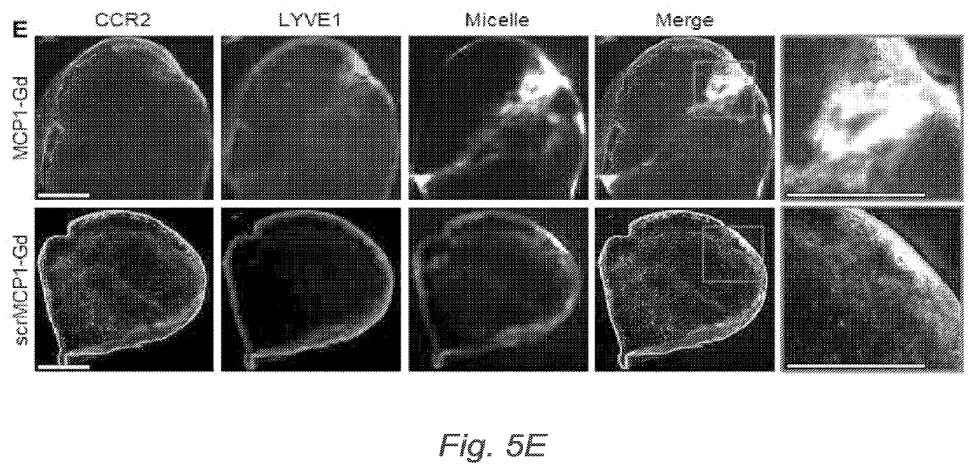

After demonstrating MCP1-Gd detection of metastatic lymph nodes (MLNs) and recurrent metastatic lymph nodes (rMLNs), it was then tested whether monocyte hitchhiking improved the MCP1-Gd targeting of MLNs in vivo. Flow cytometry confirmed that MLNs contained approximately 80% more CD11b+/CCR2+ inflammatory monocytes than CLNs, corroborating studies that reported increased monocyte recruitment to LNs under inflammatory conditions (FIG. 5A, p<0.01). (50,51) Then, monocytes in C57BL/6 mice were depleted through intraperitoneal injections of the CCR2 antagonist RS102895 to elucidate the role and contribution of monocyte hitchhiking on LN targeting (FIG. 5B). Flow cytometry indicated >75% reduction in CCR2+ cells following RS102895 treatment, confirming monocyte depletion. After injection of MCP1-Gd, ex vivo fluorescence imaging indicated ~70% reduction in LN targeting in monocyte-depleted mice (FIGS. 5C, p<0.01). However, no difference was observed in the scrMCP1-Gd group, which demonstrates that molecular targeting of CCR2 facilitates monocyte hitchhiking and enhances LN delivery. In addition, flow cytometry indicated that monocyte depletion reduced the percentage of LN cells bound with MCP1-Gd from 37.9% to ~2% (p<0.005), while no change was observed in scrMCP1-Gd, further indicating that CCR2 targeting facilitates LN accumulation (FIG. 5D). Given that monocytes enter the lymph nodes (LNs) through high endothelial venules (HEVs) in the inner cortex, it was next investigated whether monocyte-hitchhiking affected micelle distribution within the LN. (52) LNs from mice injected with MCP1-Gd or scrMCP1-Gd were sectioned and immunostained for CCR2, as well as LYVE1, which identified lymphatic endothelial cells located in the outer capsule of LNs (FIG. 5E).48% of micelle signal was observed in the outer region of the LN in the MCP1-Gd group, increasing to 65% in the scrMCP1-Gd group, indicating that molecular targeting of CCR2 led to increased MCP1-Gd delivery into the LN cortex, where cells of the adaptive immunity are located. In totality, these mechanistic studies identified monocyte hitchhiking as the primary mechanism for MCP1-Gd accumulation in the LNs and corroborated recent studies reporting the potential of immune cell hitchhiking for enhancing nanoparticle delivery. (53,54)

### In Vivo Safety and Biocompatibility of MCP1-Gd

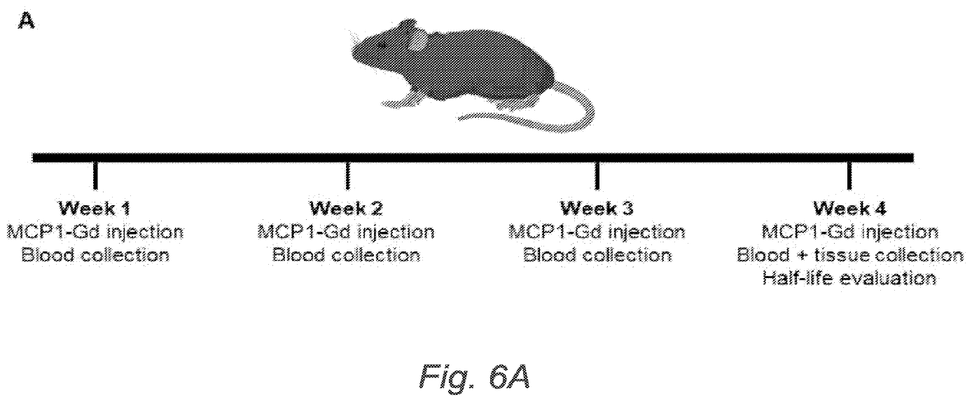

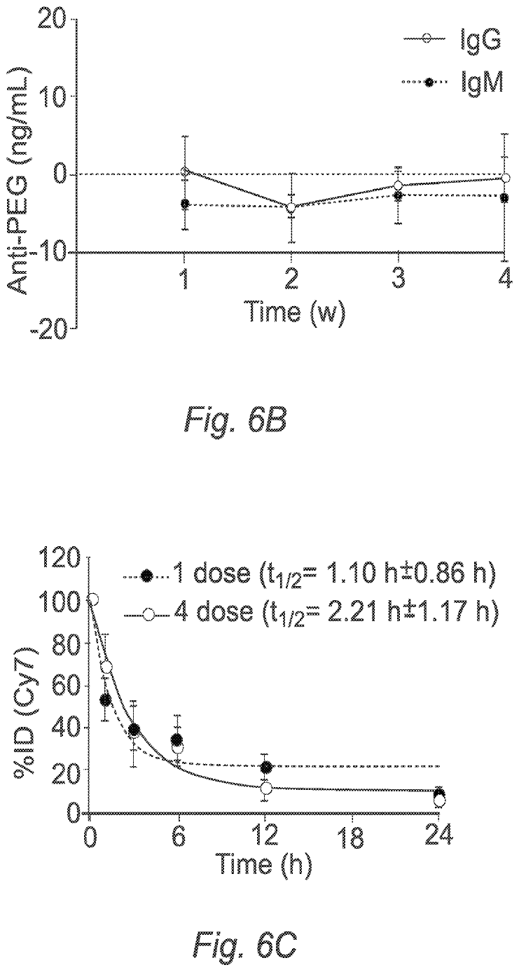

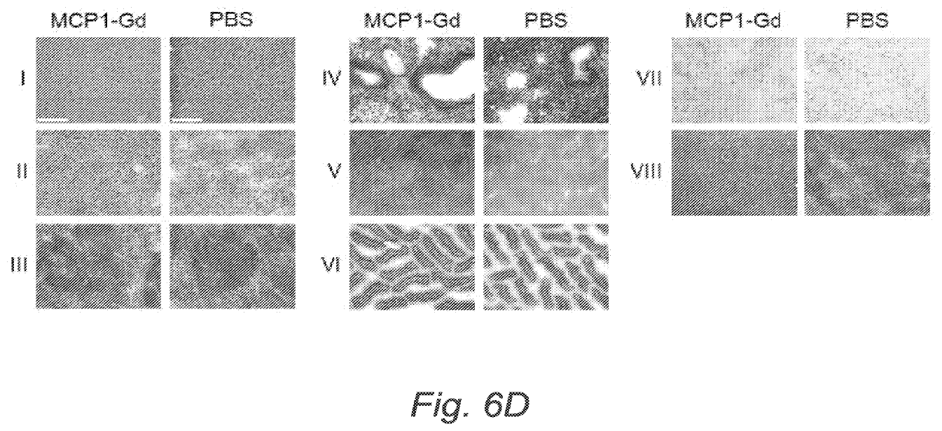

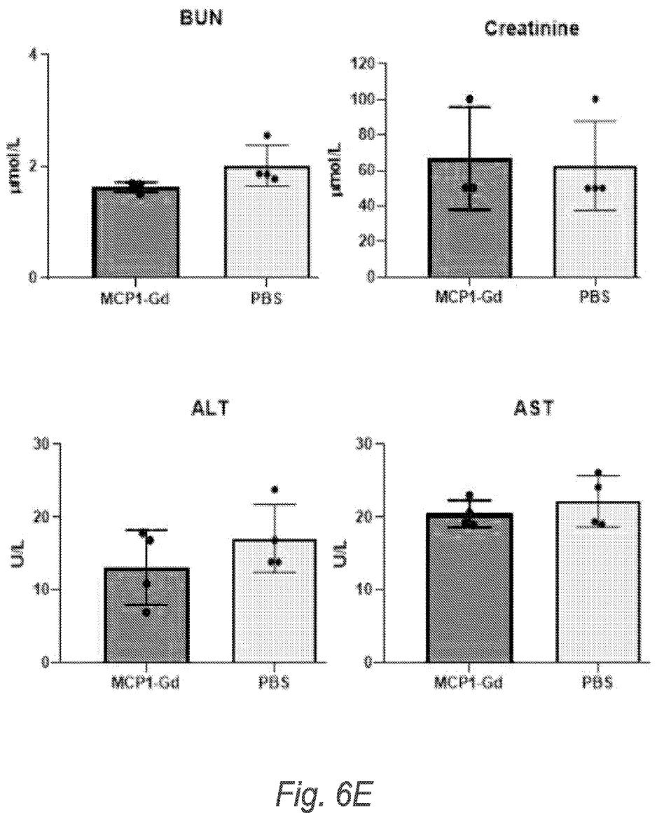

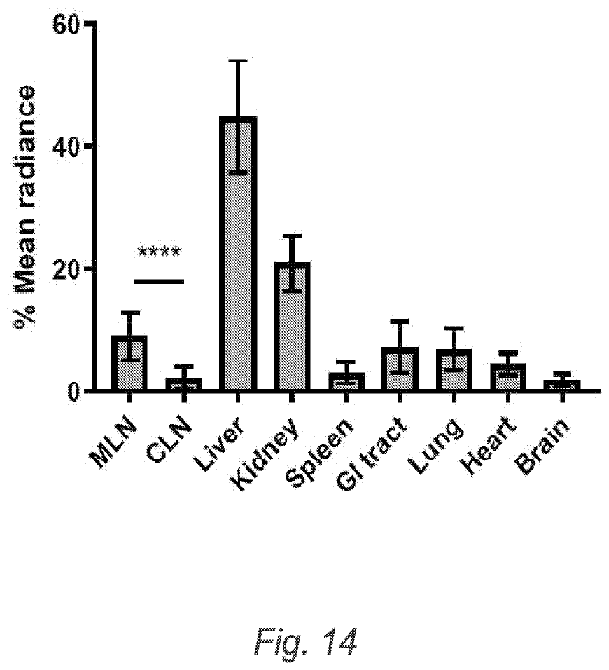

LN screenings for metastatic recurrence are performed every 3 to 12 months following surgery, depending on the size and thickness of the primary tumor. (55) Hence, the diagnostic tools used in these screenings must be safe for repeated use without inducing toxicity. Thus, the in vivo immunogenicity, plasma half-life, and biocompatibility of MCP1-Gd was characterized over the course of 4 weeks following weekly MCP1-Gd doses (FIG. 6A). Recent literature has reported the development of anti-PEG immunogenic responses following repeated exposure of PEGylated biomaterials, resulting in accelerated blood clearance, reduced efficacy, and detrimental hypersensitivity reactions. (56-58) Hence, the potential immunogenicity and characterized the plasma half-life of MCP1-Gd in mice before and after exposure to MCP1-Gd were tested. As shown in FIG. 6B, anti-PEG IgG and IgM titers remained unchanged over the duration of MCP1-Gd exposure, indicating that MCP1-Gd does not induce an immunogenic response against PEG. Then, the plasma half-life of MCP1-Gd in naïve mice were evaluated, as well as in mice that had been exposed to 3 prior doses of MCP1-Gd, as accelerated blood clearance of nanomaterials can indicate immunogenic response and reduced efficacy (FIG. 6C). (59) No difference was observed in the plasma half-lives of MCP1-Gd in naïve and pre-exposed mice, further indicating the safety of repeated MCP1-Gd injection. MCP1-Gd clearance was also calculated, and while clearance in the pre-exposed mice increased ~22% compared to naïve mice, this difference did not reach statistical significance (0.204 vs 0.167 mL/h, p=0.238). In addition, hematoxylin and eosin (H&E)-stained tissues indicated no morphological damage associated with repeated MCP1-Gd dosing, further corroborating its safety as an MRI contrast agent (FIG. 6D). Because clinical gadolinium contrast agents like Magnevist have been reported to induce nephrotoxicity even after a single dose, renal toxicity assays were performed using plasma from mice treated with four weekly doses of MCP1-Gd (FIG. 6E). (60,61) In addition, hepatic toxicity was also evaluated, since PAMs are also cleared by the liver (FIG. 14). (62) No differences were observed in the renal (BUN & creatinine) and hepatic (ALT & AST) health markers between mice treated with MCP1-Gd or phosphate-buffered saline (PBS), confirming the long-term safety of MCP1-Gd.

### Experimental Limitations

Toward clinical translation as a diagnostic tool, additional characterization regarding the long-term stability and safety of MCP1-Gd will be needed. Although the results indicated no discernible toxicity following four weekly injections, the potential risk of Gd accumulation over more clinically relevant time scales (e.g., years) will better determine its safety profile. An additional limitation is the use of only one metastatic cancer cell line in these studies. Given the heterogeneity of cancer, the inclusion of multiple cancer models of varying CCR2 expression and metastatic profiles would strengthen the robustness and better determine the potential of MCP1-Gd as a diagnostic tool for MLN detection. Lastly, monocyte depletion was achieved via the treatment of mice with CCR2 antagonists. Although CCR2 expression is highest in inflammatory monocytes, it is also expressed in subsets of T cells, dendritic cells, and endothelial cells, and the effects of CCR2 antagonism in these cells were not evaluated.

### Conclusions

Despite the improved prognosis and survival benefit associated with chemo- and immunotherapy of lymph node (LN) metastasis and recurrence, early detection remains a significant obstacle. Current clinical tools including biopsy and MRI are limited by health risks or lack sensitivity for detecting early metastasis. (63) Targeting nanoparticles to cancer-specific biomarkers as well as to tumor-tropic monocytes has been reported to increase chemotherapy drug delivery to tumor tissues but has yet to be explored in the context of metastatic LN imaging and diagnostics. (64) In these experiments, MCP1-Gd micelles were developed for the imaging and detection of lymph node (LN) metastasis and recurrence through molecular targeting of CCR2, which enables both metastatic cancer cell binding and monocyte hitchhiking for the targeted delivery of gadolinium (Gd) to metastatic LNs. MCP1-Gd was found to be biocompatible in vitro and exhibited CCR2-specific binding and hitchhiking onto monocytes, resulting in enhanced transport across lymphatic endothelium. In vivo models of LN metastasis and LN recurrence were developed and utilized to verify MCP1-Gd targeting of metastatic LNs, which enabled earlier cancer detection than conventional MRI. Furthermore, it was demonstrated that MCP1-Gd accumulation in lymph nodes (LNs) is facilitated by circulating monocytes, as depletion of these cells in vivo resulted in a significant reduction in micelle targeting to the LNs. Given the mechanism of monocyte-targeting, it is plausible that MCP1-Gd could accumulate in tissues or pathologies characterized by high monocyte infiltration that are unrelated to lymph node metastasis, representing both a potential for use in broader diagnostic applications as well as a potential for false positives. Thus, additional studies examining MCP1-Gd biodistribution and accumulation within contexts of benign LN inflammation will be needed in the future to provide valuable insight into their sensitivity and capability as a diagnostic tool for recurrent LN metastasis. In summary, the potential of CCR2-targeting micelles for MRI detection of metastatic LNs is reported herein.

### Materials & Methods

### Materials and Cells

1,2-Distearoyl-sn-glycero-3phosphoethanolamine-N-[maleimide (polyethylene glycol)-2000] (DSPE-PEG2000-maleimide) and 1,2-distearoyl-sn-glycero-3phosphoethanolamine-N-[amino(polyethylene glycol)-2000] (DSPE-PEG2000-amine) were purchased from Avanti Lipids (Alabaster, AL, USA). Cy7 mono-N-hydroxysuccinimide (NHS) ester was purchased from Lumiprobe (Hunt Valley, MD, USA). DSPE-PEG2000-FITC (FITC=fluorescein isothiocyanate) was purchased from Creative PEGWorks (Durham, NC, USA). Diethylenetriamine-N,N,N″,N″-tetra-tert-butyl acetate-N′-acetic acid (DTPA-tetra(t-Bu ester)) was purchased from Macrocyclics (Plano, TX, USA). GdC13 was purchased from Sigma-Aldrich (St. Louis, MO, USA). Antibodies were purchased from Thermo Fisher (Waltham, MA, USA). Cell lines were purchased from the American Type Culture Collection (ATCC, Manassas, VA, USA). Cell culture reagents were purchased from Gibco (Waltham, MA, USA) and Sigma-Aldrich.

### Synthesis of MCP1-Gd Micelles

MCP1 [CYNFTNRKISVQRLASYRRITSSK] and scrMCP1 [CYNSLVFRIRNSTORKYRASIST] peptides were synthesized through Fmoc-mediated solid-phase peptide synthesis and cleaved from the resin using a 94:2.5:2.5:1 vol % mixture of trifluoroacetic acid, water, ethanedithiol, and triisopropylsilane. Following ether precipitation and lyophilization, crude peptides were dissolved in milli-Q (MQ) water and purified through high-performance liquid chromatography (HPLC, Prominence, Shimadzu, Columbia, MD, USA) on a Luna C18 reverse-phased column (Phenomenex, Torrance, CA, USA) at 55° C. using water and acetonitrile supplemented with 0.1% formic acid as the mobile phases. The purity of eluted peptides was characterized using matrix-assisted laser desorption/ionization time-of-flight mass spectroscopy (MALDI-TOF-MS, Bruker, MA, USA.). Following purification, peptides were lyophilized, resuspended in MQ water, and mixed with a 10% molar excess of DSPE-PEG2000-maleimide. The pH of the mixture was then adjusted to 7.2 with 1 M NaOH, incubated for 72 h at room temperature (RT) under constant agitation, purified through HPLC, and characterized via MALDI-TOF-MS.

DSPE-PEG2000-Gd amphiphiles were synthesized by first combining DTPA-tetra(t-Bu ester) and DSPE-PEG2000-amine in peptide synthesis-grade dimethylformamide (DMF). To facilitate the formation of a peptide bond, 4× and 8× mol equiv of the coupling reagent HCTU and N,N-diisopropylethylamine (DIPEA), respectively, were added under constant stirring. Additional 1× and 2× mol equiv of HCTU and DIPEA were added 12 and 24 h following the start of the reaction. Crude DSPE-PEG2000-DTPA-tetra(t-Bu ester) was isolated through ether precipitation, lyophilized, and then purified through HPLC. Following HCl-mediated t-Bu ester deprotection, Gd3+ ions from GdC13 were chelated to DSPE-PEG2000-DTPA-tetra in a 0.5 M sodium acetate (pH 5.5) solution for 1 h. Free Gd3+ ions were separated using a desalting column, and successful synthesis of DSPE-PEG2000-Gd was confirmed via MALDI-TOF-MS.

DSPE-PEG2000-Cy7 amphiphiles were synthesized by dissolving DSPE-PEG2000-amine in a 0.1 M sodium bicarbonate solution and mixing with a solution containing 3× mol equiv of Cy7 NHS ester in peptide synthesis-grade DMF (reaction volume was 90% sodium bicarbonate and 10% DMF). The resultant mixture was protected from light, stirred overnight at RT, and then purified through HPLC before characterization via MALDI-TOF-MS.

Micelles were self-assembled through thin-film hydration. (65) Briefly, MCP1, scrMCP1, DSPE-PEG2000-Gd, DSPE-PEG2000-Cy7, and DSPE-PEG2000-FITC amphiphiles were dissolved in methanol, mixed at desired molar ratios, sonicated, and placed under a gentle nitrogen stream to evaporate the methanol before hydration in MQ water, 1 mM NaCl, or PBS. The micelle formulations and the respective molar ratios used are listed in Table 1.

### Transmission Electron Microscopy (TEM)

400-mesh carbon TEM grids (Ted Pella, Redding, CA, USA) were glow-discharged before application of 5 μL of 30 μM micelles suspended in MQ water for 5 min. Samples were then wicked to remove excess liquid and washed with MQ water. Grids were stained twice with 2% uranyl acetate for 3 min before washing with MQ water. Grids were dried overnight and then imaged on an FEI Talos F200C microscope (Thermo Fisher).

### Dynamic Light Scattering (DLS)

Micelles were suspended at 25 μM in 1 mM NaCl, filtered, and then placed in a folded capillary zeta cell for size, zeta potential, and polydispersity measurements using a Zetasizer Ultra instrument (Malvern Panalytical, Malvern, UK). N=3.

### r1 Relaxivity of Micelles

T1 relaxation times of MCP1-Gd micelles (0.2, 0.4, 0.6, 0.8, and 1.0 mM Gd) were evaluated using a 7T PET-MR system (MR Solutions Ltd., Guildford, UK) at the Zilkha Neurogenetic Institute (University of Southern California, Los Angeles, CA, USA). A 9 cm diameter radiofrequency body coil was used (bore size≈24 mm, up to 600 mT m-1 maximum gradient). A T1-weighted variable flip angle fast low angle shot (FLASH) gradient echo sequence was used with the following imaging parameters on a 7T scanner to determine particle T1 relaxation times: echo time (TE)=5 ms, repetition time (TR)=60 ms, flip angle (FA) array=(15°, 30°, 45°, 70°, and 80°), field of view (FOV)=36×36 mm, slice thickness=1 mm, image matrix=192×192, number of averages (NA)=4. A multiecho multislice spine echo sequence (MEMS) was used to determine T2 relaxation times of the particle with the following parameters: ten echoes with 15 ms interval beginning with TE=15, TR=2500, FOV=36×36 mm, slice thickness=1 mm, matrix size=256×256, NA=1. Using ROCKETSHIP v.1.1 code in MATLAB (R2014b), T1 and T2 maps were generated through a pixel-by-pixel exponential fitting of signal intensities across the different FA and TE values, respectively. Regions of interest (ROIs) were manually drawn around each concentration of MCP1-Gd micelles that were measured using ImageJ. R1 maps were generated by taking the inverse of the T1. Using R1 values, r1 relaxivity was determined (R1/concentration of Gd) over Gd concentrations of 0.2, 0.4, 0.6, 0.8, and 1.0 mM.

### Cell Culture

SVEC4-10 cells were cultured in Dulbecco's Modified Eagle Medium (DMEM) with 4.5 g/L glucose supplemented with 10% fetal bovine serum (FBS) and 1% penicillin and streptomycin (PS). Cells were maintained below 80% confluency, and culture media was renewed every 2-3 days. B16F10-Luc2 cells were cultured in DMEM with 4.5 g/L glucose supplemented with 10% FBS, 1% PS, and 10 μg/mL blasticidin. Cells were maintained below 80% confluency, and culture media was renewed every 2-3 days. NCI-H460 cells were cultured in Roswell Park Memorial Institute (RPMI) 1640 medium supplemented with 10% FBS and 1% PS. Cells were maintained below 80% confluency, and culture media was renewed every 2-3 days. WEHI-274.1 cells were cultured in DMEM with 4.5 g/L glucose supplemented with 10% FBS, 0.05 mM 2-mercaptoethanol, and 1% PS. Cells were passaged every 2-3 days. All cells were cultured in a humidified incubator set to 37° C. and 5% CO₂.

### In Vitro Micelle Biocompatibility

4000 SVEC4-10 or WEHI274.1 cells were seeded into the wells of a 96-well plate and cultured overnight before the addition of MCP1-Gd or scrMCP1-Gd micelles (1, 10, or 100 μM) or PBS. After 48 h of incubation, cell viability was measured using the MTS Cell Proliferation Assay Kit from Bio Vision (Milpitas, CA, USA). Viability was calculated as a percentage by normalizing sample absorbance measurements to those of a PBS-treated control. N=6.

### In Vitro Micelle Binding (Microtiter Plate Reader)

For adherent cells (B16F10-Luc2 and NCI-H460), 10,000 cells were seeded into black 96-well plates and cultured overnight. Cells were then incubated with 25 μM of Cy7-labeled MCP1-Gd or scrMCP1-Gd micelles for 1 h at 37° C. before 2 PBS washes and quantification of fluorescence (ex./em. 750/773). For nonadherent cells (WEHI-274.1), 10,000 cells were incubated with 25 μM of Cy7-labeled MCP1-Gd or scrMCP1-Gd micelles for 1 h at 37° C. in plastic microtubes. After 2 PBS washes, cells were resuspended in 100 μL of PBS and transferred to black 96-well plates for fluorescence measurement. N=6.

### In Vitro Micelle Binding (Confocal Microscopy)

For adherent cells (B16F10-Luc2 and NCI-H460), 250,000 cells were seeded onto 22 mm×22 mm glass coverslips in 6-well plates and allowed to adhere overnight at 37° C. Then, cells were incubated with 25 μM Cy7-labeled MCP1-Gd or scrMCP1-Gd for 1 h at ° C. before 2 PBS-Tween 20 (PBST) washes and fixation with 4% paraformaldehyde (PFA) for 10 min at RT. After subsequent PBST washes, cells were blocked with 5% normal goat serum (NGS) for at least 30 min and then incubated overnight at 4° C. with a primary CCR2 antibody (Thermo Fisher PA5-23043, 1:50). Afterward, cells were washed two times with PBST and incubated with a secondary antibody conjugated with AF594 (Thermo Fisher A-11037, 1:500) for 1 h at RT. Following PBST washes, cells were counterstained with 2 μg/mL 4′,6-diamidino-2-phenylindole (DAPI) for 5 min at RT, mounted onto microscope slides using VectaMount, sealed with nail polish, and then imaged using a Zeiss LSM 880 confocal microscope (Zeiss, Oberkochen, Germany). For nonadherent cells (WEHI-274.1), 500,000 cells were incubated with 25 μM Cy7-labeled MCP1-Gd or scrMCP1-Gd in plastic microtubes for 1 h at 37° C. Cells were then washed with PBS, fixed using 4% PFA, and then spun onto 22 mm×22 mm glass coverslips at 500 g for 5 min before immunostaining and imaging as described above.

### In Vitro Micelle Hitchhiking

To develop an in vitro model of a lymphatic endothelial barrier, 24-well transwell membrane inserts with 5 μm pores were coated with 0.1% gelatin for 1 h at RT. After washing the membrane twice with PBS, 75,000 SVEC4-10 cells were seeded and cultured for 72 hours to form a monolayer. (66) Monolayer formation and barrier function was evaluated by placing FITC-dextran (molecular weight (MW) 4000) in the top chamber, incubating for 1 h at 37° C., and then quantifying FITC fluorescence in the bottom chamber (FIG. 11). Following monolayer formation, transwell systems were incubated at 37° C. for 4 h under the following 3 conditions: (1) MCP1-Gd or scrMCP1-Gd micelles in the top chamber, (2) MCP1-Gd or scrMCP1-Gd micelles and monocytes in the top chamber, and (3) MCP1-Gd or scrMCP1-Gd micelles and monocytes in the top chamber and chemoattractant (FBS) in the bottom chamber. A micelle concentration of 25 μM was used for the hitchhiking experiments. After incubation, media was sampled from the bottom chamber to evaluate micelle transport through Cy7 fluorescence and monocyte migration through double-stranded DNA (dsDNA) quantification using the Quant-iT PicoGreen assay (Thermo Fisher) (FIG. 12). N=3.

### In Vivo Metastatic Lymph Node Mouse Model

To develop an in vivo metastatic lymph node model, 100 μL of a 1:1 mixture of Matrigel (Corning, Corning, NY, USA) and PBS containing 200,000 luciferase-expressing B16F10-Luc2 cells was injected into the flanks of C57BL/6 mice. After 2 weeks, mice were injected intraperitoneal (i.p.) with 200 μL of a 15 mg/mL suspension of D-luciferin in sterile PBS, and bioluminescence imaging was performed every 3-5 min to determine the kinetic profile of luciferase activity and optimal time point for dosage (FIG. 13). Mice were injected with D-luciferin and euthanized 2 weeks after tumor inoculation, ipsilateral and contralateral inguinal lymph nodes were collected, and bioluminescence imaging was performed to evaluate metastasis. In addition, lymph nodes were processed into single-cell suspensions and cultured for 2 weeks in culture media containing 10 μg/mL blasticidin to determine the presence of B16F10-Luc2 cells within the lymph nodes.

### In Vivo Lymph Node Recurrence Model

To simulate lymph node recurrence following surgical resection of a primary tumor, the mouse model described above was first developed, and then the primary tumor was surgically removed 12 days after tumor implantation using a linear incision. Wounds were sutured shut using nonadsorbable nylon sutures. Mice were then euthanized 7 d postsurgery to evaluate the presence of cancer in the LNs through bioluminescent imaging.

### In Vivo Lymph Node Accumulation of MCP1-Gd

Metastatic lymph node models or lymph node recurrence models were established as described above, and then 100 μL of 2.2 mM Cy7-labeled MCP1-Gd was injected via the tail-vein. MRI scans were taken 3 h postinjection of micelles as described below. Mice were euthanized immediately following the MRI scans, and their lymph nodes and other tissues were collected for subsequent ex vivo imaging and histology. A clinically used formulation of DTPA-Gd, by Magnevist, was used as a control contrast agent. N=3 or 6.

### MRI Scans

Mice were anesthetized with 2.5% isoflurane at a flow rate of 250 μL/min, before transfer onto a heated scanner bed with isoflurane levels between 1.5% and 2%. Temperature was monitored and maintained at 37° C. An electrocardiogram (ECG) was monitored and used for gating by inserting two leads subcutaneously on the anterior side close to each axilla and inserting another lead subcutaneously on the lower right abdomen. A pneumatic pillow was placed underneath the mice for respiration monitoring and gating. The mice were positioned at the magnet bore isocenter by using a motorized system. Temperature, respiration, and ECG were monitored by using SAII equipment and accompanying PC-SAM software.

A bird cage whole mouse body coil, with an axial field of view of 60 cm and a 35 mm inner diameter, was used during ECG and respiration-gated cardiac imaging. A gradient echo scan was used to obtain three orthogonal slices for positioning. A series of multi slice T1-weighted FSE (TE=11 ms, TR=1500 ms, FA=90°, FOV=32×32 mm, slice thickness=0.5 mm, matrix size=192×192, NA=2) sequences were used to image the inguinal lymph nodes. ImageJ was used to measure T1 signal intensity and calculate signal-to-noise ratios (SNRs).

### Histology

Following dissection, tissues (liver, kidneys, spleen, lungs, heart, small intestines, brain, and inguinal lymph nodes) were embedded in an optimal cutting temperature (OCT) compound (Sakura Finetek, Torrance, CA, USA) and snap-frozen using isopentane and liquid nitrogen. Then, frozen tissues were cryosectioned into 10 μm slices using a CM3050 S Cryostat (Leica, Nussloch, Germany). Tissue sections were stained with hematoxylin and eosin or processed for immunohistochemical (IHC) staining and imaging. For IHC staining, slides were fixed in 10% neutral-buffered formalin, washed with TBST, blocked with 5% NGS, immunostained for CCR2 (1:50), Ki67 (1:200), CD11b (1:100), LYVE1 (1:50), antirat Alexa Fluor 488 secondary antibodies (1:500), and/or antirabbit Alexa Fluor 594 secondary antibodies (1:500), counterstained with 2 μg/mL DAPI, and imaged using a DMi8 inverted fluorescence microscope (LEICA, Wetzlar, Germany).

### In Vivo Monocyte Depletion

The CCR2 antagonist RS102895 was dissolved in dimethyl sulfoxide (DMSO) at a concentration of 12.5 mg/mL and then diluted 20× in 0.9% saline. Mice were injected i.p. with 200 μL of the diluted RS102895 solution every 6 h for 18 h (4 total injections) to deplete monocytes. (67) Twenty-four hours after the first RS102895 injection, 100 μL of 2.2 mM FITC- or Cy7-labeled MCP1-Gd or scrMCP1-Gd were injected via the tail-vein, and mice were euthanized 3 h postinjection. Tissues including the inguinal lymph nodes were collected for ex vivo optical imaging, flow cytometry, or immunofluorescent staining. Control mice were injected with PBS.

### Flow Cytometry

All flow cytometry experiments were run on a CytoFLEX cytometer (Beckman Coulter, Brea, CA, USA) and analyzed using FlowJo. The flow cytometry buffer used was sterile PBS supplemented with 0.1% bovine serum albumin (BSA) and 1 mM ethylenediaminetetraacetic acid (EDTA). Tissue samples were prepared by rough-cutting the tissues with sterile scissors and gently pushing the resultant pieces through 70-μm cell strainers to yield single-cell suspensions that were then centrifuged at 500 g for 5 min at RT. After discarding the supernatant, the cell pellet was resuspended in 100 μL of flow cytometry buffer and immunostained for CD11b (Miltenyi 130-113-238, San Diego, CA, 1:50) and CCR2 (Miltenyi 130-119-658, 1:50) 10 min in the dark at 4° C. Samples were then washed once more with flow cytometry buffer, resuspended at 1 million cells/mL, and analyzed in the flow cytometer. All samples were confirmed to have >80% viability through 0.4% Trypan Blue staining prior to flow cytometry. The gating strategy is as follows: forward scatter (FSC) versus side scatter (SSC) plots were used to exclude debris from the analysis, and singlets were selected via FSC-H vs FSC-A plots. Then, inflammatory monocytes were identified by gating for CCR2+ and CD11b+ cells.

### In Vivo Immunogenicity

C57BL/6 mice were injected once per week with 100 μL of 2.2 mM Cy7-labeled MCP1-Gd intravenously (i.v.) through the tail-vein for 4 weeks (4 total injections). Blood samples were collected via tail-vein bleed prior to each injection using heparinized capillary tubes. Plasma was collected by spinning blood samples at 1000 g for 10 min at 4° C. and collecting the supernatant. Plasma samples were then assayed for anti-PEG IgG and IgM via ELISA according to the manufacturer instructions.

### Plasma Half-Life and Clearance

Blood samples from naïve mice or mice injected weekly with 2.2 mM MCP1-Gd (3 prior doses) were collected via the tail-vein 5 min, 1, 3, 6, 12, and 24 h following injection with Cy7-labeled MCP1-Gd and then spun at 1000 g for 10 min at 4° C. to obtain plasma. Plasma samples were then analyzed for Cy7 fluorescence using a microplate reader to determine the MCP1-Gd half-life. MCP1-Gd clearance was calculated by dividing the injected dose by the area under the curve (AUC) of the dose-overtime plot.

### Blood Chemistry Markers of Renal and Liver Health

Plasma samples from mice dosed weekly with MCP1-Gd as described above or with PBS were evaluated for hepatic and renal health markers using ALT, AST, BUN, and creatinine kits (Sigma-Aldrich, St. Louis, MO) as directed by manufacturer instructions.

### In Vivo Biodistribution

Biodistribution was evaluated 3 h after tail-vein injection of 100 μL of Cy7-labeled MCP1-Gd or Magnevist (450 μM Gd) via fluorescence imaging or inductively coupled plasma-optical emission spectroscopy (ICP-OES) (Thermo Fisher). Tissues including the liver, kidneys, spleen, lungs, heart, gastrointestinal (GI) tract, brain, and inguinal lymph nodes were dissected following euthanasia and imaged ex vivo for Cy7 fluorescence. Fluorescence data are expressed as % mean radiance.

### Statistical Analysis

Data are expressed as mean±standard deviation (SD). Statistical analysis between two groups was performed using a Student's t-test. Comparisons of three or means were performed through analysis of variance (ANOVA) followed by posthoc Tukey's tests for multiple comparisons. A p-value<0.05 was considered to be statistically significant.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Zhou, H., P. J. Lei, and T. P. Padera, Progression of Metastasis through Lymphatic System. Cells, 2021. 10(3).
2. Reticker-Flynn, N. E., et al., Lymph node colonization induces tumor-immune tolerance to promote distant metastasis. Cell, 2022. 185(11): p. 1924-1942 e23.
3. Pagliarulo, V., et al., Detection of occult lymph node metastases in locally advanced node-negative prostate cancer. J Clin Oncol, 2006. 24(18): p. 2735-42.
4. Wong, S. L., et al., Sentinel Lymph Node Biopsy and Management of Regional Lymph Nodes in Melanoma: American Society of Clinical Oncology and Society of Surgical Oncology Clinical Practice Guideline Update. J Clin Oncol, 2018. 36 (4): p. 399-413.
5. Roaten, J. B., et al., Identifying risk factors for complications following sentinel lymph node biopsy for melanoma. Arch Surg, 2005. 140(1): p. 85-9.
6. Isik, A., et al., Lymphedema After Sentinel Lymph Node Biopsy: Who Is at Risk? Lymphat Res Biol, 2022. 20(2): p. 160-163.
7. Leung, A. M., et al., Staging of regional lymph nodes in melanoma: a case for including nonsentinel lymph node positivity in the American Joint Committee on Cancer staging system. JAMA Surg, 2013. 148(9): p. 879-84.
8. Faries, M. B., et al., Lymph node metastasis in melanoma: a debate on the significance of nodal metastases, conditional survival analysis and clinical trials. Clin Exp Metastasis, 2018. 35(5-6): p. 431-442.
9. de Bondt, R. B., et al., Morphological MRI criteria improve the detection of lymph node metastases in head and neck squamous cell carcinoma: multivariate logistic regression analysis of MRI features of cervical lymph nodes. Eur Radiol, 2009. 19(3): p. 626-33.
10. Park, S. Y., et al., Prediction of Micrometastasis (<1 cm) to Pelvic Lymph Nodes in Prostate Cancer: Role of Preoperative MRI. AJR Am J Roentgenol, 2015. 205(3): p. W328-34.
11. Giannarini, G., G. Petralia, and H. C. Thoeny, Potential and limitations of diffusion-weighted magnetic resonance imaging in kidney, prostate, and bladder cancer including pelvic lymph node staging: a critical analysis of the literature. Eur Urol, 2012. 61(2): p. 326-40.
12. Hovels, A. M., et al., The diagnostic accuracy of C T and MRI in the staging of pelvic lymph nodes in patients with prostate cancer: a meta-analysis. Clin Radiol, 2008. 63(4): p. 387-95.
13. Dulinska-Litewka, J., et al., Superparamagnetic Iron Oxide Nanoparticles-Current and Prospective Medical Applications. Materials (Basel), 2019. 12(4).
14. Weissleder, R., et al., Ultrasmall superparamagnetic iron oxide: an intravenous contrast agent for assessing lymph nodes with M R imaging. Radiology, 1990. 175(2): p. 494-8.
15. Czarniecki, M., et al., Ultra-small superparamagnetic iron oxide contrast agents for lymph node staging of high-risk prostate cancer. Transl Androl Urol, 2018. 7(Suppl 4): p. S453-S461.
16. Daldrup-Link, H. E., Ten Things You Might Not Know about Iron Oxide Nanoparticles. Radiology, 2017. 284(3): p. 616-629.
17. Harisinghani, M. G., et al., Noninvasive detection of clinically occult lymph-node metastases in prostate cancer. N Engl J Med, 2003. 348(25): p. 2491-9.
18. Wang, Y. X. and J. M. Idee, A comprehensive literatures update of clinical researches of superparamagnetic resonance iron oxide nanoparticles for magnetic resonance imaging. Quant Imaging Med Surg, 2017. 7(1): p. 88-122.
19. Singh, N., et al., Potential toxicity of superparamagnetic iron oxide nanoparticles (SPION). Nano Rev, 2010. 1.
20. Heesakkers, R. A., et al., Prostate cancer: detection of lymph node metastases outside the routine surgical area with ferumoxtran-10-enhanced M R imaging. Radiology, 2009. 251(2): p. 408-14.
21. Wang, Y. X., Superparamagnetic iron oxide based MRI contrast agents: Current status of clinical application. Quant Imaging Med Surg, 2011. 1(1): p. 35-40.
22. Chin, D. D., et al., Collagenase-Cleavable Peptide Amphiphile Micelles as a Novel Theranostic Strategy in Atherosclerosis. Adv Ther (Weinh), 2020. 3(3).
23. Poon, C., M. Sarkar, and E. J. Chung, Synthesis of Monocyte-targeting Peptide Amphiphile Micelles for Imaging of Atherosclerosis. J Vis Exp, 2017(129).
24. Chyu, K. Y., et al., Immunization using ApoB-100 peptide-linked nanoparticles reduces atherosclerosis. JCI Insight, 2022. 7(11).
25. Poon, C., et al., Hybrid, metal oxide-peptide amphiphile micelles for molecular magnetic resonance imaging of atherosclerosis. J Nanobiotechnology, 2018. 16(1): p. 92.
26. Chung, E. J., et al., In vivo biodistribution and clearance of peptide amphiphile micelles. Nanomedicine, 2015. 11(2): p. 479-87.
27. Xu, M., et al., Role of the CCL2-CCR2 signalling axis in cancer: Mechanisms and therapeutic targeting. Cell Prolif, 2021. 54(10): p. e13115.
28. Verweij, S. L., et al., CCR2 expression on circulating monocytes is associated with arterial wall inflammation assessed by 18F-FDG PET/C T in patients at risk for cardiovascular disease. Cardiovasc Res, 2018. 114(3): p. 468-475.
29. Huang, Y., et al., The effect of size, charge, and peptide ligand length on kidney targeting by small, organic nanoparticles. Bioeng Transl Med, 2020. 5(3): p. e10173.
30. Liu, H., et al., Preparation, characterization, in vivo pharmacokinetics, and biodistribution of polymeric micellar dimethoxycurcumin for tumor targeting. Int J Nanomedicine, 2015. 10: p. 6395-410.
31. Xiong, Y., et al., A robust in vitro model for trans-lymphatic endothelial migration. Sci Rep, 2017. 7(1): p. 1633.
32. Thomas, S. N. and A. Schudel, Overcoming transport barriers for interstitial-, lymphatic-, and lymph node-targeted drug delivery. Curr Opin Chem Eng, 2015. 7: p. 65-74.
33. Archer, P. A., et al., Quantitation of lymphatic transport mechanism and barrier influences on lymph node-resident leukocyte access to lymph-borne macromolecules and drug delivery systems. Drug Deliv Transl Res, 2021. 11(6): p. 2328-2343.
34. Schudel, A., D. M. Francis, and S. N. Thomas, Material design for lymph node drug delivery. Nat Rev Mater, 2019. 4(6): p. 415-428.
35. Nakamura, T., et al., The Effect of Size and Charge of Lipid Nanoparticles Prepared by Microfluidic Mixing on Their Lymph Node Transitivity and Distribution. Mol Pharm, 2020. 17(3): p. 944-953.
36. Yoo, S. P., et al., Gadolinium-Functionalized Peptide Amphiphile Micelles for Multimodal Imaging of Atherosclerotic Lesions. ACS Omega, 2016. 1(5): p. 996-1003.
37. An, J., et al., Targeting CCR2 with its antagonist suppresses viability, motility and invasion by downregulating MMP-9 expression in non-small cell lung cancer cells. Oncotarget, 2017. 8(24): p. 39230-39240.
38. Jakubzick, C., et al., Minimal differentiation of classical monocytes as they survey steady-state tissues and transport antigen to lymph nodes. Immunity, 2013. 39(3): p. 599-610.
39. Randolph, G. J., Emigration of monocyte-derived cells to lymph nodes during resolution of inflammation and its failure in atherosclerosis. Curr Opin Lipidol, 2008. 19(5): p. 462-8.
40. Lund, H., et al., Transient Migration of Large Numbers of CD14 (++) CD16 (+) Monocytes to the Draining Lymph Node after Onset of Inflammation. Front Immunol, 2016. 7: p. 322.
41. Mitchell, L. A., et al., Optimized dosing of a CCR2 antagonist for amplification of vaccine immunity. Int Immunopharmacol, 2013. 15(2): p. 357-63.
42. von Schuckmann, L. A., et al., Risk of Melanoma Recurrence After Diagnosis of a High-Risk Primary Tumor. JAMA Dermatol, 2019. 155(6): p. 688-693.

1. An MRI contrast delivery system comprising:
a plurality of nanoparticles wherein each nanoparticle includes a targeting peptide that target CCR2 receptors conjugated thereto and an MRI contrast moiety conjugated thereto.

2. The MRI contrast delivery system of claim 1, wherein the nanoparticles are configured to hitchhike on monocytes for targeted delivery to metastatic lymph nodes, thereby enhancing MRI signal specificity and sensitivity.

3. The MRI contrast delivery system of claim 1, wherein the targeting peptides include CCR2 receptor-targeting peptides.

4. The MRI contrast delivery system of claim 2, wherein the CCR2 receptor-targeting peptide is a fragment of a MCP1.

5. The MRI contrast delivery system of claim 2, wherein the CCR2 receptor-targeting peptide is a fragment of a peptide having sequence
(SEQ ID NO: 1)
QPDAINAPVTCCYNFTNRKISVQRLASYRRITSSKCPKEAVIFK
TIVAKEICADPKQKWVQDSMDHLDKQTQTPKT.

6. The MRI contrast delivery system of claim 2, wherein the CCR2 receptor-targeting peptide is a peptide having CYNFTNRKISVQRLASYRRITSSK (SEQ ID NO: 2) or a peptide thereof with 1 to 7 conservative substitutions.

7. The MRI contrast delivery system of claim 1, wherein the plurality of nanoparticles includes nanoparticles selected from the group consisting of micelles, liposomes, lipid nanoparticles, and combinations thereof.

8. The MRI contrast delivery system of claim 1, wherein the plurality of nanoparticles includes a plurality of micelles.

9. The MRI contrast delivery system of claim 1, wherein the plurality of nanoparticles includes a plurality of unilamellar micelles.

10. The MRI contrast delivery system of claim 1, wherein the MRI contrast moiety includes gadolinium.

11. The MRI contrast delivery system of claim 1, wherein the MRI contrast moiety includes iron oxide.

12. The MRI contrast delivery system of claim 1, wherein the MRI contrast moiety includes manganese.

13. The MRI contrast delivery system of claim 1 wherein the targeting peptides and the MRI contrast moiety are independently connected to the nanoparticles by reaction with a functional group.

14. The MRI contrast delivery system of claim 13, wherein the functional group that can be used for linking includes amines, carboxylic acids, NHS esters, acid anhydrides, or unsaturated imides (e.g., maleimide).

15. The MRI contrast delivery system of claim 13, wherein the functional group for the MRI contrast moiety including a chelating moiety.

16. The MRI contrast delivery system of claim 1, wherein plurality of nanoparticles includes a plurality of micelles, each micelle including a plurality of targeting peptide-conjugated amphiphiles, a plurality of MRI contrast moiety-conjugated amphiphiles, and an optional plurality of non-targeted amphiphiles.

17. The MRI contrast delivery system of claim 16, wherein the plurality of targeting peptide-conjugated amphiphiles includes amphiphiles having a first phospholipid conjugated to a CCR2 receptor-targeting peptide with a first linking group, the plurality of MRI contrast moiety-conjugated amphiphiles includes amphiphiles having a second phospholipid conjugated to the MRI contrast moiety with a second linking group, and the optional plurality of non-targeted amphiphiles includes amphiphiles having a third phospholipid conjugated to a capping moiety with a third linking group.

18. The MRI contrast delivery system of claim 17, wherein the first linking group, the second linking group, and the third linking group are each independently a polyethylene glycol having a weight average molecular weight from about 500 to 10000 Daltons.

19. The MRI contrast delivery system of claim 16, wherein the first phospholipid, the second phospholipid, and the third phospholipid are each independently selected from the group consisting of phosphatidic acids, phosphatidyl inositols, phosphatidyl cholines, phosphatidyl ethanolamines, phosphatidyl serines, phosphatidyl glycerols, and any combinations thereof.

20. The MRI contrast delivery system of claim 16, wherein the first phospholipid, the second phospholipid, and the third phospholipid are each independently selected from the group consisting of phosphatidylglycerol, lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine, palmitoyloleoylphosphatidylcholine, di stearoylphosphatidylcholine, dioleoylphosphatidylcholine, dipalmitoylphosphatidylcholine, dioleoylphosphatidylglycerol, dipalmitoylphosphatidylglycerol, -phosphatidylethanolamine, dioleoyl-phosphatidylethanolamine 4-(N-maleimidomethyl)-cyclohexane-1-carboxylate (DOPE-mal), 1-stearoyl-2-oleoyl phosphatidylcholine, 1,2-distearoyl-sn-glycerol-3-phosphoethanolamine, and combinations thereof.

21. The MRI contrast delivery system of claim 16 further comprising a pharmaceutically acceptable carrier.

22. The MRI contrast delivery system of claim 21 further comprising a chemotherapeutic agent conjugated to the nanoparticles or encapsulated by the nanoparticles.

23. A method for imaging lymph node metastasis, the method comprising:
administering the MRI contrast delivery system described of claim 1 to a subject at risk for having lymph node metastasis; and
taking MRI images of the subject to determine the presence of lymph node metastasis.

24. The method of claim 23, wherein the lymph node metastasis includes micro metastasis.

25. A method for detecting lymph node (LN) metastasis in a subject using magnetic resonance imaging (MRI), the method comprising:
administering to the subject a composition comprising nanoparticles incorporating gadolinium (Gd) and peptides derived from the CCR2-binding motif of MCP1;
allowing for the migration of monocytes hitchhiked by the micelles to metastatic LNs; and
conducting MRI to detect the enhanced signal from the metastatic LNs due to the accumulation of the gadolinium-incorporating micelles or fragments therefrom.

26. The method of claim 25, wherein the detecting step comprises quantifying the MRI signal enhancement in the LNs, thereby providing a measure of metastatic burden.

27. The method of claim 25, wherein the peptide synthesis involves solid-phase peptide synthesis techniques.

28. The method of claim 25 further comprising a step of purifying the peptide using high-performance liquid chromatography (HPLC).

29. The method of claim 25, wherein the conjugation of the peptide to the lipid tail includes a maleimide-functionalized lipid.

30. The method of claim 25 further comprising a step of characterizing the micelles using dynamic light scattering to determine their size.
